(19) United States
(12) Patent Application Publication
(10) Pub. No.: US 2025/0235553 A1
(43) Pub. Date: Jul. 24, 2025

(54) ABHD18 inhibitors

(71) Applicant: Scenic Biotech B.V., Amsterdam (NL)

(72) Inventors: Anchal Srivastava, Amsterdam (NL); Bastiaan Evers, Amsterdam (NL); Sebastian M.B. Nijman, Amsterdam (NL); Vincent Arthur Blomen, Amsterdam (NL); Linda M.M. Clijsters, Amsterdam (NL)

(73) Assignee: Scenic Biotech B.V., Amstedam (NL)

(21) Appl. No.: 18/703,367

(22) PCT Filed: Oct. 20, 2022
(86) PCT No.: PCT/EP2022/079269

§ 371 Date: Apr. 22, 2024

Related U.S. Application Data
(60) Provisional application No. 63/271,020, filed on Oct. 22, 2021.

(51) Int. Cl. A61K 48/00; A61K 45/06; A61P 9/00

(52) U.S. Cl. CPC A61K 48/005; A61K 45/06; A61P 9/00

(57) ABSTRACT

The current invention provides ABHD18 inhibitors, ABHD18 inhibitors for therapeutic use, related compositions and related assays.

### FIELD

The invention relates to the field of protein inhibitors, specifically ABHD18 inhibitors, their therapeutic uses and compositions.

### BACKGROUND OF THE INVENTION

As described in the review by Dudek (Front. Cell Dev. Biol., 29 Sep. 2017), “The phospholipid cardiolipin (CL) is an essential constituent of mitochondrial membranes and plays a role in many mitochondrial processes, including respiration and energy conversion. Pathological changes in CL amount or species composition can have deleterious consequences for mitochondrial function and trigger the production of reactive oxygen species.” Disorders caused by a dysfunctional cardiolipin metabolism, by CL dysfunction, by a dysfunctional CL pool and/or by changes in CL amount and/or species composition are rare diseases often eluding effective treatments (see again the review by Dudek, supra, and the further review by Wasmus and Dudek, Life 2020, 10, 277; doi: 10.3390/life10110277, as well as the further references cited in these reviews).

Diseases and disorders that are caused by, associated with and/or characterized by dysfunctional cardiolipin metabolism (including dysfunctional cardiolipin biosynthesis and/or remodeling), by CL dysfunction, by a dysfunctional CL pool and/or by changes in CL amount and/or species composition are also generally referred to herein as a “cardiolipin disorder”, and examples thereof will be clear to the skilled person based on the further disclosure and references cited herein.

The present invention generally relates to methods for preventing and/or treating (as defined herein) cardiolipin disorders (as defined herein).

In a further aspect, the invention relates to compounds and compositions that can be used in the prevention and/or treatment of cardiolipin disorders (as defined herein), and to the use of such compounds and compositions for treating cardiolipin disorders. As further described herein, such compounds and compositions may in particular be (compositions comprising) an inhibitor of the protein called ABHD18, and more in particular an inhibitor of the enzymatic activity of the ABHD18 protein, and even more in particular an inhibitor of at least one catalytic activity of the ABHD18 protein, all as further described herein. In particular, said enzymatic and/or catalytic activity of the ABHD18 protein may be an activity that ABHD18 exerts on at least one cardiolipin in a cell or in the body of the subject to be treated, and/or on the cardiolipin metabolism in said subject, again as further described herein.

Such methods, compounds, compositions and uses can all be as further described herein.

In a further aspect, the cardiolipin disorder that is prevented and/or treated in accordance with the present invention is chosen from the group consisting of a mitochondrial disorder (as described herein, and in particular a mitochondrial respiratory disorder as described herein, and more in particular Barth syndrome), antiphospholipid syndrome (APS), diabetic cardiomyopathy (DCM), a kidney disorder (as described herein, and in particular a kidney disorder induced by obesity and/or high fat diets, again as further described herein), an immunological disorder (as further described herein), cancer, Parkinson's disease, ischemia reperfusion injury, heart failure, traumatic brain injury or ageing.

As a prime example of a cardiolipin disorder, Barth syndrome is a heritable cardiolipin remodeling disorder, caused by mutations in the TAZ gene (tafazzin; Xq28), which encodes taffazin, an acyltransferase involved in the remodeling of cardiolipin. The current standard care involves the administration of agents such as angiotensin-converting enzyme inhibitors, angiotensin II, receptor blockers, beta-blockers and diuretics to combat cardiac symptoms, and granulocyte-colony-stimulating factor to treat neutropenia (Finsterer, J. (2019). Barth syndrome: mechanisms and management. The application of clinical genetics, 12, 95.).

However, the standard treatment for Barth syndrome remains restricted to the (partial) relief of symptoms, without addressing the underlying mechanism of defective cardiolipin remodeling. Moreover, despite the administration of G-CSF and the normalization of the granulocyte count, patients often still develop severe infections.

Hence, there is an unmet need in the art for an effective treatment for Barth syndrome, preferably addressing its underlying causes.

Thus, in a further aspect, the present invention generally relates to methods for preventing and/or treating a mitochondrial disorder, and in particular a mitochondrial respiratory disorder (as described herein), and even more in particular Barth syndrome, to compounds and compositions that can be used in the prevention and/or treatment of these disorders, and to the use of such compounds and compositions for preventing and/or treating these disorders. Again, according to a particular aspect, such compounds and compositions are (compositions comprising) an inhibitor of the protein called ABHD18, and more in particular an inhibitor of the enzymatic activity of the ABHD18 protein, and even more in particular an inhibitor of at least one catalytic activity of the ABHD18 protein, all as further described herein. In particular, said enzymatic and/or catalytic activity of the ABHD18 protein may be an activity that ABHD18 exerts on at least one cardiolipin in a cell or in the body of the subject to be treated, and/or on the cardiolipin metabolism in said subject, again as further described herein.

Other aspects, embodiments, advantages, uses and applications of the present invention will become clear from the further description herein.

### DESCRIPTION OF THE INVENTION

### ABHD18 Inhibitors

In a first aspect, the invention provides an inhibitor of an ABHD18 protein, wherein said inhibitor is able to decrease an activity of said ABHD18 protein. Such inhibitors are referred to in the current application as inhibitors according to or of the invention.

In specific aspects, an inhibitor according to the invention is for use as a medicament, preferably for use in the treatment and/or prevention of a disease disclosed herein, more preferably for use in the treatment and/or prevention of a cardiolipin disorder, most preferably for use in the treatment and/or prevention of Barth syndrome.

### Structure of ABHD18 Proteins

An ABHD18 protein, which may also be called an abhydrolase domain-containing protein 18 or an alpha/beta hydrolase domain-containing protein 18, is a protein encoded by a ABHD18 gene. Herein, all human variants and isoforms, and species homologues and their variants and isoforms are encompassed. In the context of this application, an ABHD18 or an FLJ21106 refer to a ABHD18 protein.

In specific aspects, an ABHD18 gene is located at chromosome 4 open reading frame 29 (C4orf29). In this context, an ABHD18 (protein) may also be called an C4orf29 (protein).

In specific aspects, an ABHD18 protein is represented by an amino acid sequence having at least 75%, 75.5%, 76%, 76.5%, 77%, 77.5%, 78%, 78.5%, 79%, 79.5%, 80%, 80.5%, 81%, 81.5%, 82%, 82.5%, 83%, 83.5%, 84%, 84.5%, 85%, 85.5%, 86%, 86.5%, 87%, 87.5%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% sequence identity with SEQ ID NO: 1, 2, 3, 4, 5 or 6 preferably with SEQ ID NO:1.

In specific aspects, an ABHD18 protein is represented by an amino acid sequence having at least 60% sequence identity with SEQ ID NO: 1, 2, 3, 4, 5 or 6. In specific aspects, an ABHD18 protein is represented by an amino acid sequence having at least 70% sequence identity with SEQ ID NO: 1, 2, 3, 4, 5 or 6. In specific aspects, an ABHD18 protein is represented by an amino acid sequence having at least 80% sequence identity with SEQ ID NO: 1, 2, 3, 4, 5 or 6. In specific aspects, an ABHD18 protein is represented by an amino acid sequence having at least 90% sequence identity with SEQ ID NO: 1, 2, 3, 4, 5 or 6. In specific aspects, an ABHD18 protein is represented by an amino acid sequence having at least 95% sequence identity with SEQ ID NO: 1, 2, 3, 4, 5 or 6.

In specific aspects, an ABHD18 protein comprises an amino acid sequence represented by SEQ ID NO: 1, 2, 3, 4, 5 or 6. In more specific aspects, an ABHD18 protein consists of an amino acid sequence represented by SEQ ID NO: 1, 2, 3, 4, 5 or 6.

In specific aspects, an ABHD18 protein has a length from 200 up to 480 amino acids, or from 210 up to 430 amino acids, or from 220 up to 430 amino acids, or from 230 up to 430 amino acids, or from 240 up to 430 amino acids, or from 250 up to 430 amino acids, or from 260 up to 430 amino acids, or from 270 up to 430 amino acids, or from 280 up to 430 amino acids, or from 290 up to 430 amino acids, or from 300 up to 430 amino acids, or from 310 up to 430 amino acids, or from 320 up to 430 amino acids, or from 330 up to 430 amino acids, or from 340 up to 430 amino acids, or from 350 up to 430 amino acids, or from 360 up to 430 amino acids, or from 370 up to 430 amino acids, or from 380 up to 430 amino acids, or from 390 up to 430 amino acids, or from 400 up to 430 amino acids, or from 410 up to 430 amino acids.

In specific aspects, an ABHD18 protein has a length from 404 up to 424 amino acids, or from 322 up to 342 amino acids, or from 311 up to 331 amino acids, or from 454 up to 474 amino acids, or from 372 up to 392 amino acids, or from 469 up to 489 amino acids. In more specific aspects, an ABHD18 protein has a length of 414 amino acids, or 332 amino acids, or 321 amino acids, or 464 amino acids, or 382 amino acids, or 479 amino acids.

In specific aspects, an ABHD18 protein is expressed in or is derived from a vertebrate, more preferably a mammal, even more preferably a rat, a mouse, a rabbit or a human, most preferably a human. In this context, an ABHD18 protein derived from a specific animal may be a recombinant protein expressed in a host organism.

In specific aspects, an ABHD18 protein comprises a hydrolase domain, preferably an alpha/beta hydrolase domain, which may also be called an abhydrolase domain.

### Activity of ABHD18 Proteins

An inhibitor according to the invention is able to inhibit (i.e. decrease) an activity of an ABHD18 protein. Herein, decreasing an activity may mean inhibiting an activity of said ABHD18 protein via direct or indirect contact between said inhibitor and said ABHD18 protein, and/or decreasing the level of expression of said ABHD18 protein thereby decreasing the total activity of said ABHD18 protein. In specific aspects, decreasing an activity means inhibiting an activity of said ABHD18 protein via direct or indirect contact between said inhibitor and said ABHD18 protein.

In specific aspects is provided an inhibitor according to the invention, wherein said inhibitor is able to decrease a catalytic activity of an ABHD18 protein. In this context, an ABHD18 protein may be called an enzyme. It is understood that a catalytic activity of an ABHD18 protein or enzyme means that said ABHD18 protein increases the rate of a reaction, preferably by a factor of at least 10, 15, 20, 25, 30, 35, 40, 45, 50, 55, 60, 65, 70, 75, 80, 85, 90, 95, 100, 200, 300, 400, 500, 600, 700, 800, 900 or 1000, relative to the rate of a corresponding reaction performed in a corresponding environment (e.g. similar cellular conditions, pH, salt concentrations, enzyme concentrations, etc.) and under corresponding conditions (e.g. same temperature, etc.) wherein said ABHD18 protein is not present. Preferably, said rate increase of said reaction is defined under physiological conditions. In the context of this application, “a catalytic activity of an ABHD18 protein, wherein said catalytic activity comprises a reaction”, “a reaction catalysed by an ABHD18 protein” or similar phrases mean that the rate of said reaction is increased by said ABHD18 Protein, as explained above.

In specific aspects, decreasing a catalytic activity means decreasing the rate of a reaction catalysed by said ABHD18 protein, more preferably by a factor equal to or lower than 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002 or 0.001, relative to the rate of a corresponding reaction performed in a corresponding environment (e.g. similar cellular conditions, pH, salt concentrations, ABHD18 concentration, other enzyme concentrations, etc.) and under corresponding conditions (e.g. same temperature, etc.) wherein said inhibitor according to the invention is not present. Preferably, said rate decrease of said reaction is defined under physiological conditions. As explained above, decreasing the rate of a reaction catalysed by an ABHD18 protein may be the result of inhibiting a catalytic activity of said ABHD18 protein via direct or indirect contact between said inhibitor and said ABHD18 protein, and/or decreasing the level of expression of said ABHD18 protein thereby decreasing the total catalytic activity of said ABHD18 protein. Preferably, decreasing the rate of a reaction catalysed by an ABHD18 protein is the results of inhibiting a catalytic activity of said ABHD18 protein via direct or indirect contact between said inhibitor and said ABHD18 protein. The rate of a reaction catalysed by a ABHD18 protein may be measured or assessed by any suitable methods well-known in the art.

In specific aspects, the rate of a reaction catalysed by a ABHD18 protein is measured or defined under physiological conditions. More preferably, said reaction takes place in an organelle, a cell fraction, a cell, a tissue, an organ or a subject, most preferably in a vertebrate, mammalian or human cell.

Without being limited to any specific explanation, mechanism or hypothesis, an inhibitor as used in the invention is capable, in a suitable assay or model and/or upon administration to a subject, of decreasing in specific aspects a catalytic activity of an ABHD18 protein that originates from, is caused by or is linked to a catalytic triad comprised in said ABHD18 protein, in particular a catalytic triad consisting of a histidine, a aspartic acid and a serine residue, and more in particular the catalytic triad that consists of His-402, Asp-375 and Ser-199.

Without being limited to any specific explanation, mechanism or hypothesis, an inhibitor as used in the invention is capable, in a suitable assay or model and/or upon administration to a subject, of decreasing the hydrolase activity of ABHD18 (or at least one hydrolase activity of ABHD18, i.e. in respect of at least one substrate of ABHD18). Again without being limited to any specific explanation, mechanism or hypothesis, an inhibitor as used in the invention is capable, in a suitable assay or model and/or upon administration to a subject, of decreasing the hydrolase activity of ABHD18 in respect of at least one cardiolipin.

In specific aspects, a catalytic activity of an ABHD18 protein comprises the cleavage of a fatty acid residue from a compound, which may also be called a deacylase activity. More preferably, said compound is a lipid and said catalytic activity comprises a lipid deacylase activity. Most preferably, said lipid is a cardiolipin and said catalytic activity comprises a cardiolipin deacylase activity.

In specific aspects, a catalytic activity of an ABHD18 protein comprises the transfer of a fatty acid residue between two compounds, which may also be called a transacylase activity. More preferably, at least one of these compounds is a lipid and said catalytic activity comprises a lipid transacylase activity. Most preferably, said lipid is a cardiolipin and said catalytic activity comprises a cardiolipin transacylase activity.

In specific aspects, an activity of an ABHD18 protein comprises a catalytic activity during lipid metabolism. Herein, it is understood that lipid metabolism comprises lipid anabolism and lipid catabolism more specific aspects, an activity of an ABHD18 protein comprises a catalytic activity during lipid synthesis. In this context, an ABHD18 protein having a catalytic activity during lipid metabolism or synthesis means that a reaction involved in lipid metabolism or synthesis is catalysed by said ABHD18 protein.

As further described herein, according to a specific aspect of the invention, an activity of an ABHD18 protein comprises an enzymatic activity in respect of at least one cardiolipin (wherein said activity is as further described herein) and/or at least one enzymatic activity of ABHD18 that is involved in cardiolipin remodeling (wherein said activity is again as further described herein).

### Structure of Inhibitors

In specific aspects, an inhibitor according to the invention is able to decrease an activity of an ABHD18 protein by inhibiting an activity of said ABHD18 protein via direct or indirect contact between said inhibitor and said ABHD18 protein. More preferably, said direct contact is non-covalent or covalent binding between said inhibitor and said ABHD18 protein.

In specific aspects, an inhibitor according to the invention is a small molecule, an antibody fragment, an antibody or an aptamer, more preferably a small molecule, an antibody fragment or an aptamer.

In specific aspects, an inhibitor according to the invention is a small molecule. Examples of inhibitors which are able to decrease an activity of an ABHD protein are described in Bononi, Giulia, et al. “α/β-Hydrolase Domain (ABHD) Inhibitors as New Potential Therapeutic Options against Lipid-Related Diseases.” Journal of Medicinal Chemistry (2021). In specific aspects, an inhibitor according to the invention is a compound described in this reference.

An antibody refers to polyclonal antibodies, monoclonal antibodies, humanized antibodies, single-chain antibodies, and fragments thereof such as Fab F (ab)2, Fv, VHH and other fragments that retain the antigen binding function of the parent antibody. As such, an antibody may refer to an immunoglobulin or glycoprotein, or fragment or portion thereof, or to a construct comprising an antigen-binding portion comprised within a modified immunoglobulin-like framework, or to an antigen-binding portion comprised within a construct comprising a non-immunoglobulin-like framework or scaffold. A fragment of an antibody refers to a part of an antibody that retain the antigen binding function of said antibody. An antigen in the context of the current invention is preferably an ABHD18 protein.

In specific aspects, an antibody or a fragment thereof is an intrabody. An intrabody is defined herein as an antibody that performs its antigen binding function intracellularly by binding to an intracellular antigen. More preferably, the intrabody is expressed in the cell wherein said antigen binding function takes place. Most preferably, the intrabody is expressed from a sequence which has been introduced by means of a viral vector into the cell.

In specific aspects, an inhibitor according to the invention is able to specifically bind said ABHD18 protein, preferably wherein the binding site comprises at least part of a hydrolase domain of said ABHD18 protein, more preferably wherein said inhibitor is a small molecule, an antibody fragment, or an aptamer. Preferably, said hydrolase domain is represented by an amino acid sequence having 80%, 80.5%, 81%, 81.5%, 82%, 82.5%, 83%, 83.5%, 84%, 84.5%, 85%, 85.5%, 86%, 86.5%, 87%, 87.5%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% sequence identity with SEQ ID NO: 4.

In specific aspects, an inhibitor according to the invention is a competitive inhibitor of said ABHD18 protein, wherein said inhibitor is able to specifically bind an active site of said ABHD18 protein associated with said activity of said ABHD18 protein.

In specific aspects, an inhibitor according to the invention is a non-competitive or allosteric inhibitor of said ABHD18 protein, wherein said inhibitor is able to specifically bind a part of said ABHD18 protein which is not an active site associated with said activity of said ABHD18 protein. In this context, said binding site which is not an active site maybe called an allosteric site.

In specific aspects, an inhibitor according to the invention is able to decrease an activity of an ABHD18 protein by decreasing the level of expression of an active form of said ABHD18 protein, preferably said expression is in a vertebrate, more preferably in a mammal, even more preferably in a rat, a mouse, a rabbit or a human, most preferably in a human. In vitro assays can be performed with representative animal models, to determine if an inhibitor according to the invention exerts the desired effect of decreasing the level of expression of said active form of said ABHD18 protein. Preferably, the level of expression of said active form of said ABHD18 protein is decreased by a factor equal to or lower than 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01. In these aspects, it is understood that an active form an ABHD18 protein may have undergone post-translational modifications.

In specific aspects, an inhibitor according to the invention is a double-stranded RNA molecule, a small inhibitory RNA (siRNA) molecule, or an inhibitory RNA molecule (RNAi), a guideRNA (gRNA). More preferably, an inhibitor according to these specific aspects are able to decrease an activity of an ABHD18 protein by decreasing the level of expression of said ABHD18 protein. In other words, these inhibitors preferably do not bind an ABHD18 protein but lead to a decreased concentration of ABHD18 proteins in an organelle, a cell fraction, a cell, tissue, organ or subject when they are introduced therein. Most preferably, the concentration of said ABHD18 protein is decreased by a factor equal to or lower than 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01 upon introduction in said organelle, cell fraction, cell, tissue, organ or subject.

In specific aspects, an inhibitor according to the invention leads to a knockout of an ABHD18 gene when introduced in an organelle, a cell fraction, a cell, tissue, organ or subject. More preferably, a knockout of an ABHD18 gene means level of expression of said ABHD18 gene is decreased by a factor equal to or lower than 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01. The terms level of expression of a gene, its corresponding RNA transcripts and its corresponding polypeptides and proteins may be used interchangeable in the context of this application.

Again, in all cases, and irrespective of the nature of the inhibitor and the manner in which it binds to ABHD18 and/or influences ABHD18 activity or expression, the inhibitor used in the invention is preferably such that it is capable of inhibiting at least one activity of ABHD18, and preferably at least one enzymatic activity of ABHD18, such as at least one enzymatic activity of ABHD18 with respect to at least one cardiolipin (wherein said activity as further described herein) and/or at least one enzymatic activity of ABHD18 that is involved in cardiolipin remodeling (wherein said activity is again as further described herein).

### Cardiolipin-Related Activity of ABHD18 Proteins

In specific aspects, an activity of an ABHD18 protein comprises a catalytic activity during cardiolipin metabolism. In more specific aspects, an activity of an ABHD18 protein comprises a catalytic activity during cardiolipin (bio) synthesis. In this context, an ABHD18 protein having a catalytic activity during cardiolipin metabolism or synthesis means that a reaction involved in cardiolipin metabolism or synthesis is catalysed by said ABHD18 protein. Reference is again made to the reviews by Dudek and by Wasmus and Dudek (both supra) as well as the further disclosure herein.

A cardiolipin (CL) or 1,3-bis(sn-3′-phosphatidyl)-sn-glycerol is a diphosphatidylglycerol lipid comprising two phosphatidylglycerols connected with a glycerol backbone to form a dimeric structure, thus comprising four fatty acid residues.

The concentration of cardiolipins, L:D cardiolipins, mature cardiolipins, nascent cardiolipins, monolysocardiolipins, dilysocardiolipins, trilysocardiolipins or tetralysocardiolipins in a composition, an organelle, a cell fraction, a cell, a membrane, a tissue, an organ or a subject refers to the total number of cardiolipins, L:D cardiolipins, mature cardiolipins, nascent cardiolipins, monolysocardiolipins, dilysocardiolipins, trilysocardiolipins or tetralysocardiolipins, respectively, in said composition, organelle, cell fraction cell, membrane, tissue, organ or subject to the weight or volume of said composition, organelle, cell fraction, cell, membrane, tissue, organ or subject. The concentrations may be determined via the methodology outlined in Example 9.

A cardiolipin may comprise one or more types of fatty acid residues. Non-limiting examples of a type of fatty acid residues are a C14, C16, C16, C18, C20, C22, C16:1, C18:1, C18:2, C18:3, C20:1, C20:2, C20:3, C20:4, C20:5, stearoyl, oleoyl, linoleoyl and linolenoyl fatty acids. Preferably, a fatty acid residue comprised in a cardiolipin is a C16, C18, C20 or C22 fatty acid residue. In animal tissues, the most abundant fatty acid residue comprised in cardiolipins is a doubly unsaturated C18 fatty alkyl chain (C18:2 fatty acid), most commonly a linoleoyl residue. The most abundant cardiolipins in animals thus comprise four C18:2 fatty acids.

The abundance of a type of fatty acid residue of or in a cardiolipin, a composition, an organelle, a cell fraction, a cell, a membrane, a tissue, an organ or a subject refers to the molar ratio of the total number of fatty acid residues of said type comprised in all cardiolipins comprised in said cardiolipin, composition, organelle, cell fraction, cell, membrane, tissue, organ or subject to the total number of said cardiolipins times four in the context of this application. The abundances may be determined via the methodology outlined in Example 9.

The cardiolipin composition of or in a cardiolipin, a composition, an organelle, a cell fraction, a cell, a membrane, a tissue, an organ or a subject refers to the abundances of all types of fatty acid residues in said cardiolipin, composition, organelle, cell fraction, cell, membrane, tissue, organ or subject in the context of this application.

In specific aspects, an inhibitor according to the invention is able to decrease an activity of an ABHD18 protein, wherein said decreased activity is monitored by a change in the cardiolipin composition of a cardiolipin, a composition, an organelle, a cell fraction, a cell, a membrane, a tissue, an organ or a subject. Such a change may be quantitative and/or qualitative.

The average fatty acid residue desaturation of or in a cardiolipin, a composition, an organelle, a cell fraction, a cell, a membrane, a tissue, an organ or a subject refers to the ratio of the total number of carbon-carbon double bonds comprised in fatty acid residues comprised in all monolysocardiolipins, dilysocardiolipins and trilysocardiolipins, tetralysocardiolipins and cardiolipins comprised in said composition, organelle, cell fraction, cell, membrane, tissue, organ or subject to the total number of said monolysocardiolipins, dilysocardiolipins and trilysocardiolipins, tetralysocardiolipins and cardiolipins. The average fatty acid residue desaturation may be determined via the methodology outlined in Example 9.

In specific aspects, an inhibitor according to the invention is able to decrease an activity of an ABHD18 protein, wherein said activity of said ABHD18 protein comprises changing the average fatty acid residue desaturation of a cardiolipin, a composition, an organelle, a cell fraction, a cell, a membrane, a tissue, an organ or a subject.

The average fatty acid residue length of or in a cardiolipin, a composition, an organelle, a cell fraction, a cell, a membrane, a tissue, an organ or a subject refers to the ratio of the total number of carbon atoms comprised in fatty acid residues comprised in all monolysocardiolipins, dilysocardiolipins and trilysocardiolipins, tetralysocardiolipins and cardiolipins comprised in said composition, organelle, cell fraction, cell, membrane, tissue, organ or subject to the total number of said monolysocardiolipins, dilysocardiolipins and trilysocardiolipins, tetralysocardiolipins and cardiolipins. The average fatty acid residue length may be determined via the methodology outlined in Example 9.

In specific aspects, an inhibitor according to the invention is able to decrease an activity of an ABHD18 protein, wherein said activity of said ABHD18 protein comprises changing the average fatty acid residue length of a cardiolipin, a composition, an organelle, a cell fraction, a cell, a membrane, a tissue, an organ or a subject.

Each of the four fatty acid residues comprised in a cardiolipin may be removed by cleavage of a corresponding ester bond. A monolysocardiolipin (MLCL), a dilysocardiolipin (DLCL) and a trilysocardiolipin (triLCL) and tetralysocardiolipin (tetraLCL) refer to cardiolipins from which respectively one, two, three or four fatty acid residues have been removed. In the context of this application, MLCL, DLCL, TLCL and tetralysocardiolipins are not considered to be cardiolipins, et vice versa, unless explicitly mentioned otherwise.

The cardiolipin dissociation of or in a cardiolipin, a composition, an organelle, a cell fraction, a cell, a membrane, a tissue, an organ or a subject refers to the molar ratio [MLCL+DLCL×2+triLCL×3+tetraLCL×4]/[(CL+MLCL+DLCL+triLCL+tetraLCL)×4] in the context of this application, wherein MLCL, DLCL, triLCL, tetraLCL and CL are defined respectively as the total number of monolysocardiolipins, dilysocardiolipins and trilysocardiolipins, tetralysocardiolipins and cardiolipins comprised in said composition, organelle, cell fraction, cell, membrane, tissue, organ or subject. The cardiolipin dissociation may be determined via the methodology outlined in Example 9.

An L:D cardiolipin is a cardiolipin, wherein the total number of carbon atoms comprised in the fatty acids comprised in said cardiolipin is L, and wherein the total number of carbon-carbon double bonds comprised the fatty acids comprised in said cardiolipin is D. For example, a cardiolipin comprising four 18:2 fatty acid residues may be called a 72:8 cardiolipin.

The abundance of an L:D cardiolipin of or in a composition, an organelle, a cell fraction, a cell, a membrane, a tissue, an organ or a subject refers to the molar ratio of the total number of L:D cardiolipins in said composition, organelle, cell fraction, cell, membrane, tissue, organ or subject to the total number of cardiolipins comprised in said composition, organelle, cell fraction, cell, membrane, tissue, organ or subject times four in the context of this application.

An L:D monolysocardiolipin is a monolysocardiolipin, wherein the total number of carbon atoms comprised in the fatty acids comprised in said monolysocardiolipin is L, and wherein the total number of carbon-carbon double bonds comprised the fatty acids comprised in said monolysocardiolipin is D. For example, a monolysocardiolipin comprising two 18:2 and one 16:1 fatty acid residues may be called a 52:3 monolysocardiolipin.

The MLCL/CL ratio of or in a composition, an organelle, a cell fraction, a cell, a membrane, a tissue, an organ or a subject refers to the molar ratio of the total number of monolysocardiolipins to the total number of cardiolipins comprised in said composition, organelle, cell fraction, cell, membrane, tissue, organ or subject in the context of this application. The MLCL/CL ratio may also be called the (molar) concentration ratio of monolysocardiolipin (MLCL) to cardiolipin (CL) in a composition, organelle, cell fraction, cell, membrane, tissue, organ or subject. According to this definition, a MLCL/CL ratio may be called a total MLCL/CL ratio, as it refers to a ratio of the number of monolysocardiolipins to the number of cardiolipins, irrespective of the type of fatty acid residues comprised in said monolysocardiolipins or cardiolipins. The MLCL/CL ratio may be determined via the methodology outlined in Example 9.

The L1: D1/L2: D2 MLCL/CL ratio of or in a composition, an organelle, a cell fraction, a cell, a membrane, a tissue, an organ or a subject refers to the MLCL/CL ratio of or in said composition, organelle, cell fraction, cell, membrane, tissue, organ or subject, wherein said monolysocardiolipins are L1: D1 monolysocardiolipins and said cardiolipins are L2: D2 cardiolipins.

Without being bound to this theory, the biosynthesis of a cardiolipin comprises the biosynthesis of a nascent cardiolipin (nCL), which is remodeled to a mature cardiolipin (mCL). Both nascent and mature cardiolipins are cardiolipins. Without being bound to this theory, the average fatty acid residue desaturation and length of nascent cardiolipin are lower than the average fatty acid residue desaturation and length of mature cardiolipin. Preferably, an average fatty acid residue desaturation which is lower means it is 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 units lower. Preferably, an average fatty acid residue length which is lower means it is 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 units lower.

In specific aspects, a nascent cardiolipin is a 70:4, 70:5, 70:6, 68:2, 68:3 or 68:4 cardiolipin. In specific aspects, a mature cardiolipin is a 72:7, 72:8, 72:9, 74:8, 74:9, or 74:10 cardiolipin, more preferably a 72:7, or 72:8 cardiolipin.

The MLCL/nCL ratio of or in a composition, an organelle, a cell fraction, a cell, a membrane, a tissue, an organ or a subject refers to the MLCL/CL ratio of or in said composition, organelle, cell fraction, cell, membrane, tissue, organ or subject, wherein said cardiolipins are nascent cardiolipins. In other words, an MLCL/nCL ratio is defined as a concentration ratio of monolysocardiolipin (MLCL) to nascent cardiolipin (CL).

The MLCL/mCL ratio of or in a composition, an organelle, a cell fraction, a cell, a membrane, a tissue, an organ or a subject refers to the MLCL/CL ratio of or in said composition, organelle, cell fraction, cell, membrane, tissue, organ or subject, wherein said cardiolipins are mature cardiolipins. In other words, an MLCL/mCL ratio is defined as a concentration ratio of monolysocardiolipin (MLCL) to mature cardiolipin (CL). In specific aspects, an MLCL/mCL ratio is an 52:2/72:8 MLCL/CL ratio.

In specific aspects, an inhibitor according to the invention is able to decrease an activity of an ABHD18 protein, wherein said activity comprises the conversion of a nascent cardiolipin to a mature cardiolipin.

Cardiolipin remodeling is the conversion of a nascent cardiolipin to a mature cardiolipin. Cardiolipin remodeling comprises a deacylation of a nascent cardiolipin to form a monolysocardiolipin (MLCL), followed by reacylation of said monolysocardiolipin to form a cardiolipin. After multiple deacylation-reacylation cycles, also known as the Lands cycles, a mature cardiolipin is formed. The key enzymes catalyzing these deacylation-reacylation cycles are a phospholipase, catalyzing the deacylation reactions, and taffazin (TAZ1), catalyzing the reacylation reactions and coded by a TAZ or Xq28 gene. In yeast, said phospholipase has been identified as cardiolipin-specific deacylase 1 (Cld1).

Again, for a further description of cardiolipins, cardiolipin metabolism and of the physiological and pathological mechanisms and effects in which these are involved, reference is made to the reviews by Dudek and by Wasmus and Dudek, both supra, as well as the further references cited therein.

Without being bound to a specific theory, explanation, mechanism or hypothesis, ABHD18 may be identified by the current invention as a phospholipase involved in cardiolipin remodeling and/or as a homologue of Cld1. Preferably, ABHD18 may function as said phospholipase in cardiolipin remodeling and/or as a homologue of Cld1 in vertebrates, more preferably in mammals, most preferably in humans.

A skilled person confronted with the knowledge that Cld1 is a phospholipase involved in cardiolipin remodeling, would not realize or be tempted to consider ABHD18 as a human homologue based on the prior art currently available. The prior art does not contain any motivation to arrive at this conclusion, neither from a mechanistic of structural perspective.

In specific aspects, an inhibitor according to the invention is able to decrease an activity of an ABHD18 protein, wherein said activity comprises the remodeling of a cardiolipin.

In specific aspects, an inhibitor according to the invention is able to decrease an activity of an ABHD18 protein, wherein said activity comprises deacylation or transacylation of a cardiolipin.

In specific aspects, an inhibitor according to the invention is able to decrease an activity of an ABHD18 protein, wherein said activity comprises, is equivalent to, corresponds to or is similar to the catalytic activity of Cld1, more preferably wherein said catalytic activity of Cld1 comprises the remodeling of a cardiolipin and/or comprises deacylation or transacylation of a cardiolipin.

In specific aspects, an inhibitor according to the invention is able to decrease an activity of an ABHD18 protein, wherein introducing said inhibitor in a composition, an organelle, a cell fraction, a cell, a membrane, a tissue, an organ or a subject is able to decrease the cardiolipin dissociation ratio of said composition, organelle, cell fraction, cell, membrane, tissue, organ or subject, preferably by a factor equal to or lower than 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01.

In specific aspects, an inhibitor according to the invention is able to decrease an activity of an ABHD18 protein, wherein introducing said inhibitor in a composition, an organelle, a cell fraction, a cell, a membrane, a tissue, an organ or a subject is able to decrease the MLCL/CL ratio of said composition, organelle, cell fraction, cell, membrane, tissue, organ or subject, preferably by a factor equal to or lower than 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01. More preferably, said MLCL/CL ratio is an MLCL/nCL ratio. Alternatively, said MLCL/CL ratio is an MLCL/mCL ratio, most preferably a 52:2/72:8 MLCL/CL ratio.

Furthermore, an unsaturated fatty acid residue comprised in a cardiolipin may undergo oxidation. Oxidation in this context is the reaction of molecular oxygen with said unsaturated fatty acid to form. Preferably, oxidation of an unsaturated fatty acid comprises the formation of a peroxidized fatty acid, which may react further to a variety of other compounds such as hydrocarbons, aldehydes, ketones, alcohols, esters and acids. An oxidized cardiolipin in this context is a cardiolipin comprising an oxidized fatty acid residue.

The cardiolipin oxidation of or in a cardiolipin, a composition, an organelle, a cell fraction, a cell, a membrane, a tissue, an organ or a subject refers to the molar ratio of the total number of oxidized fatty acids comprised in a cardiolipin, a monolysocardiolipin, a dilysocardiolipins or a trilysocardiolipins over the total number of fatty acids comprised in monolysocardiolipins, dilysocardiolipins and trilysocardiolipins and cardiolipins.

In specific aspects, an inhibitor according to the invention is able to decrease an activity of an ABHD18 protein, wherein said activity of said ABHD18 protein comprises changing the cardiolipin oxidation of a cardiolipin, a composition, an organelle, a cell fraction, a cell, a membrane, a tissue, an organ or a subject.

A cardiolipin may be degraded, meaning that it can be converted into one or more compounds that are not cardiolipins in a reaction that is irreversible under physiological conditions. Without being bound to this theory, an oxidized cardiolipin may be more prone to degradation than a cardiolipin which is not oxidized.

In specific aspects, an inhibitor according to the invention is able to induce one or more of the following changes in a composition, an organelle, a cell fraction, a cell, a membrane, a tissue, an organ or a subject, when it is introduced therein:
an altered average fatty acid residue length; and/or
an altered cardiolipin dissociation, preferably a decreased cardiolipin dissociation, more preferably wherein the cardiolipin dissociation is decreased by a factor equal to or lower than 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01; and/or
an altered MLCL/CL ratio, preferably a decreased MLCL/CL ratio, more preferably wherein the MLCL/CL ratio is decreased by a factor equal to or lower than 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01, most preferably wherein said MLCL/CL ratio is an MLCL/nCL ratio or an MLCL/mCL ratio; and/or
an altered cardiolipin concentration, preferably an increased cardiolipin concentration, more preferably an increased nascent cardiolipin concentration, most preferably wherein the concentration is increased by a factor equal to or greater than 1.5, 1.6, 1.7, 1.8, 1.9, 2, 2.5, 3, 3.5, 4, 4.5, 5, 5.5, 6, 6.5, 7, 7.5, 8, 8.5, 9, 9.5 10, 15, 20, 25, 30, 35, 40, 45, 50, 55, 60, 65, 70, 75, 80, 85, 90, 95, 100; and/or
an altered 70:4, 70:5, 70:6, 68:2, 68:3 and/or 68:4 cardiolipin concentration, preferably an increased concentration, more preferably wherein the concentration is increased by a factor equal to or greater than 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5 10, 15, 20, 25, 30, 35, 40, 45, 50, 55, 60, 65, 70, 75, 80, 85, 90, 95, 100; and/or
an altered cardiolipin oxidation, preferably an decreased cardiolipin oxidation, more preferably wherein the cardiolipin oxidation is decreased by a factor equal to or lower than 0.85, 0.8, 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01; and/or
an altered cardiolipin composition; and/or
an altered average fatty acid residue desaturation;
wherein said alterations, increases and decreases are relative to the corresponding parameter as measured under essentially the same conditions but without the presence of (e.g. before introduction of) said inhibitor according to the invention. Again, said parameter(s) may be measured in the manner described herein (preferred), or alternatively in a manner known per se which will be clear to the skilled person based on the disclosure herein.

Cardiolipins form an important component of the inner mitochondrial membrane, where they constitutes about 20% of the total lipid composition. In mammalian cells, cardiolipins are found almost exclusively in the inner mitochondrial membrane where they are essential for the optimal function of enzymes involved in mitochondrial metabolism.

Cardiolipins are important for the structure and the function of the inner mitochondrial membrane and the mitochondrial electron transport chain supercomplexes comprised therein. Without being limiting, cardiolipins serve an important function in the mitochondrial membrane by (i) serving as a proton trap, (ii) regulating, facilitating and/or participating in the physical structure of the membrane and complexes comprised therein and (iii) playing a role in apoptosis.

First, cardiolipins comprised in the inner mitochondrial membrane may function as proton traps during oxidative phosphorylation. Each of the two phosphates in cardiolipin can capture one proton. Although it has a symmetric structure, ionization of one phosphate happens at different levels of acidity than ionizing both, with pK₁=3 and pK₂>7.5. Hence, under normal physiological conditions, a cardiolipin may carry only one negative charge. Hydroxyl groups on the phosphate form stable intramolecular hydrogen bonds, forming a bicyclic resonance structure. This structure traps one proton, which is conducive to oxidative phosphorylation.

During the oxidative phosphorylation process catalyzed by Complex IV, large quantities of protons are transferred from one side of the membrane to another side causing a large pH change. Without wishing to be bound by theory, it has been suggested that cardiolipin functions as a proton trap within the mitochondrial membranes, strictly localizing the proton pool and minimizing pH in the mitochondrial intermembrane space. This function is thought to be due to the unique structure of cardiolipin, which, as described above, can trap a proton within the bicyclic structure while carrying a negative charge. Thus, cardiolipin can serve as an electron buffer pool to release or absorb protons to maintain the pH near the mitochondrial membranes.

Second, cardiolipins may regulate, facilitate and/or participate in the physical structure of the inner mitochondrial membrane.

Specifically, it may be involved in regulating the physical properties of the inner mitochondrial membrane and/or regulating, facilitating and/or participating in aggregate and quaternary structures due to its unique lipid structure. Inter alia, cardiolipins may influence membrane fluidity and osmotic stability, and may participate in protein function via direct interaction with membrane proteins. Cardiolipin has been found in tight association with inner membrane protein complexes such as the cytochrome bcl complex (complex III). As well, it has been localized to the contact sites of dimeric cytochrome c oxidase, and cardiolipin binding sites have also been found in the ADP/ATP carrier (AAC).

Furthermore, cardiolipin may play a role in the formation of respiratory chain supercomplexes (respirasomes), also called mitochondrial electron transport chain supercomplexes.

Third, cardiolipins comprised in the inner mitochondrial membrane may play a role in apoptosis. An early event in the apoptosis cascade involves cardiolipin. A cardiolipin-specific oxygenase produces cardiolipin-hydroperoxides which causes the lipid to undergo a conformational change. The oxidized cardiolipin then translocates from the inner mitochondrial membrane to the outer mitochondrial membrane where it is thought to form a pore through which cytochrome c is released into the cytosol. Cytochrome c can bind to the IP3 receptor stimulating calcium release, which further promotes the release of cytochrome c. When the cytoplasmic calcium concentration reaches a toxic level, the cell dies. In addition, extra-mitochondrial cytochrome c interacts with apoptotic activating factors, causing the formation of apoptosomal complexes and activation of the proteolytic caspase cascade.

### Cardiolipin (Remodeling) Disorders

In the context of this application, a cardiolipin disorder is a disease or a disorder caused by, related to and/or characterized by a dysfunctional cardiolipin metabolism (including but not limited to dysfunctional cardiolipin remodeling), by CL dysfunction and/or a dysfunctional CL pool, and/or by changes in CL amount and/or species composition. A cardiolipin disorder is preferably a cardiolipin remodeling disorder, wherein a cardiolipin remodeling disorder is caused by, related to and/or characterized by a dysfunctional cardiolipin modeling. It should be noted that, in its broadest sense, the present invention is not limited to any underlying cause for the cardiolipin disorder, the dysfunctional cardiolipin metabolism or remodeling), the CL or CL pool dysfunction or the undesired CL amount and/or species composition, as long as the inhibitor of the invention is capable of treating or preventing (as further described herein) the disease or disorder to be treated, alleviating at least one symptom thereof, restoring or partially restoring at least one disease-relevant parameter (as further described herein), and/or preventing a further progression of the disease be treated and/or a further deterioration of at least one such symptom or disease-relevant parameter. For example, and without limitation, the underlying cause could be a defect in the activity and/or amount of ABHD18 protein that is present in said subject (e.g. expressed by the cells of said subject) or a defect in any other protein, enzyme or gene that is present in and/or expressed by the subject to be treated, such as another protein, enzyme or gene that is involved in CL biosynthesis, metabolism and/or remodeling.

In specific aspects, an inhibitor according to the invention is for use in the treatment and/or prevention of a cardiolipin disorder, preferably a cardiolipin remodeling disorder.

In specific aspects is provided an inhibitor of an ABHD18 protein according to the invention, wherein said ABHD18 protein is expressed in an subject suffering from a cardiolipin disorder, preferably a cardiolipin remodeling disorder, or wherein said ABHD18 protein is expressed in an organelle, a cell fraction, a cell, tissue or organ derived from such a subject. Preferably, said inhibitor is for use in the treatment and/or prevention of a cardiolipin disorder, preferably a cardiolipin remodeling disorder.

According to a preferred definition, a cardiolipin disorder or a cardiolipin remodeling disorder is a disorder characterized by one or more of the following altered parameters:
an altered cardiolipin composition; and/or
an altered concentration of mature cardiolipins, preferably a decreased concentration of mature cardiolipins, more preferably wherein the concentration of mature cardiolipins is decreased by a factor equal to or lower than 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01; and/or
an altered concentration of 72:7 and/or 72:8 cardiolipins, preferably a decreased concentration, more preferably wherein the concentration is decreased by a factor equal to or lower than 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01; and/or
an altered average fatty acid residue length; and/or
an altered average fatty acid residue length, preferably a decreased average fatty acid residue desaturation, preferably wherein the average fatty acid residue length is decreased by a factor equal to or lower than 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01; and/or
an altered cardiolipin dissociation, preferably an increased cardiolipin dissociation, more preferably wherein the cardiolipin dissociation is increased by a factor equal to or greater than 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5 10, 15, 20, 25, 30, 35, 40, 45, 50, 55, 60, 65, 70, 75, 80, 85, 90, 95, 100; and/or
an altered MLCL/CL ratio, preferably an increased MLCL/CL ratio, more preferably wherein the MLCL/CL ratio is increased by a factor equal to or greater than 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5 10, 15, 20, 25, 30, 35, 40, 45, 50, 55, 60, 65, 70, 75, 80, 85, 90, 95, 100, most preferably wherein said MLCL/CL ratio is an MLCL/nCL ratio or an MLCL/mCL ratio; and/or
an altered cardiolipin oxidation, preferably an increased cardiolipin oxidation, more preferably wherein the cardiolipin oxidation is increased by a factor equal to or greater than 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5 10, 15, 20, 25, 30, 35, 40, 45, 50, 55, 60, 65, 70, 75, 80, 85, 90, 95, 100; and/or
an altered structure and/or function of an inner mitochondrial membrane; and/or
an altered formation and/or structure and/or function of a mitochondrial electron transport chain supercomplex;
wherein said parameters are determined in an organelle, a cell fraction, a cell, a tissue, an organ or a subject, wherein said alterations, increases and decreases are relative to a corresponding organelle, cell fraction, cell, tissue organ derived from a healthy subject or to a corresponding healthy subject, preferably wherein said parameters are determined in a muscle tissue or a muscle cell.

Without being bound by this theory, these altered parameters may disrupt or decrease, partially or entirely, the function of cardiolipins comprised in the inner mitochondrial membrane as described above. Inter alia, these altered parameters may disrupt or decrease, partially or entirely, cardiolipins (i) serving as a proton trap, (ii) regulating, facilitating and/or participating in the physical structure of the membrane and complexes comprised therein and (iii) playing a role in apoptosis.

In the context of this application, a healthy subject is a subject not suffering from a cardiolipin (remodeling) disorder such as Barth syndrome. Preferably, said subject is a mammal, more preferably said subject is a human.

In specific aspects, an inhibitor according to the invention is able to mitigate one or more of the altered parameters described above after introduction in a subject suffering from a cardiolipin disorder or a cardiolipin remodeling disorder.

In specific aspects, an inhibitor of the invention is able to induce one or more of the following changes when introduced in a subject suffering from a cardiolipin disorder, preferably a cardiolipin remodeling disorder, or in a corresponding composition, membrane, organelle, cell fraction, cell, tissue or organ:
a restored or partially restored average fatty acid residue length; and/or
a restored or partially restored cardiolipin dissociation, preferably wherein the cardiolipin dissociation is decreased after introduction of the inhibitor, more preferably by a factor equal to or lower than 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01; and/or
a restored or partially restored MLCL/CL ratio, preferably wherein the MLCL/CL ratio is decreased after introduction of the inhibitor, more preferably by a factor equal to or lower than 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01, most preferably wherein said MLCL/CL ratio is an MLCL/nCL ratio or an MLCL/mCL ratio; and/or
a restored or partially restored cardiolipin concentration, preferably wherein the cardiolipin concentration is increased after introduction of the inhibitor, more preferably by a factor equal to or greater than 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5 10, 15, 20, 25, 30, 35, 40, 45, 50, 55, 60, 65, 70, 75, 80, 85, 90, 95, 100; and/or
an altered nascent cardiolipin concentration, preferably an altered 70:4, 70:5, 70:6, 68:2, 68:3 and/or 68:4 cardiolipin concentration, preferably wherein the concentration is increased after introduction of the inhibitor, more preferably by a factor equal to or greater than 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5 10, 15, 20, 25, 30, 35, 40, 45, 50, 55, 60, 65, 70, 75, 80, 85, 90, 95, 100; and/or
a restored or partially restored cardiolipin oxidation, preferably wherein the cardiolipin oxidation is decreased after introduction of the inhibitor, more preferably by a factor equal to or lower than 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01; and/or
a restored or partially restored structure and/or function of an inner mitochondrial membrane comprised in said subject, composition, membrane, organelle, cell fraction, cell, tissue or organ; and/or
a restored or partially restored formation and/or structure and/or function of a mitochondrial electron transport chain supercomplexes comprised in said subject, composition, membrane, organelle, cell fraction, cell, tissue or organ; and/or
a restored or partially restored cardiolipin composition; and/or
a restored or partially restored average fatty acid residue desaturation;
wherein a partially restored parameter means that the alteration of said parameter relative to a corresponding composition, membrane, organelle, cell fraction, cell, tissue or organ derived from a healthy subject or to a corresponding healthy subject is significantly smaller after introduction of said inhibitor,
wherein a restored parameter means that said parameter is essentially the same as in a corresponding composition, organelle, cell fraction, cell, membrane, tissue or organ derived from a healthy subject or as in a corresponding healthy subject,
preferably wherein said tissue is a muscle tissue, or wherein said cell is a muscle cell, or wherein said organelle is a mitochondrion,
preferably wherein said inhibitor is for use in the treatment and/or prevention of a cardiolipin disorder, preferably a cardiolipin remodeling disorder.

In particular, such a parameter can be considered “partially restored” when said parameter, following after treatment with the inhibitor in accordance with the invention, is closer in value to the same parameter in a healthy subject, compared to value of the same parameter prior to treatment with said inhibitor, and/or more generally is considered by a skilled person (and in particular, the treating physician) as an improvement in said parameter.

In the context of this invention, a corresponding composition, membrane, organelle, cell fraction, cell, tissue or organ in the context of a disorder or disease such as a cardiolipin (remodeling) disorder means a composition, membrane, organelle, cell fraction, cell, tissue or organ derived from, isolated from, or serving as in vitro or in vivo model for a subject suffering from said disorder or disease.

According to a preferred definition, a cardiolipin disorder or a cardiolipin remodeling disorder is a disorder characterized by an altered structure and/or function of the inner mitochondrial membrane, and/or altered formation and/or structure and/or function of the mitochondrial electron transport chain supercomplexes comprised therein. Herein, said alterations are relative to a corresponding membrane, organelle, cell fraction, cell, tissue or organ derived from a healthy subject or to a corresponding healthy subject.

In specific aspects, an inhibitor according to the invention is able to mitigate the altered structure and/or function of the inner mitochondrial membrane, and/or altered formation and/or structure and/or function of the mitochondrial electron transport chain supercomplexes comprised therein, as described above, after introduction in a subject suffering from a cardiolipin disorder or a cardiolipin remodeling disorder, or in a corresponding composition, membrane, organelle, cell fraction, cell, tissue or organ. Preferably said inhibitor is for use in the treatment and/or prevention of a cardiolipin disorder, preferably a cardiolipin remodeling disorder.

### Barth Syndrome

Barth syndrome is a heritable cardiolipin remodeling disorder, caused by mutations in the TAZ gene (tafazzin; Xq28), which encodes taffazin, an acyltransferase involved in the remodeling of cardiolipin, as explained above. Defective or reduced taffazin function results in abnormal remodeling of cardiolipin and compromises mitochondrial structure and respiratory chain function. Taffazin is expressed at high levels in cardiac and skeletal muscle and is involved in the maintenance of the inner membrane of mitochondria. Taffazin is involved in maintaining levels of cardiolipin, which is essential for energy production in the mitochondria. Illustrative sequences of TAZ1 isoforms are given by, for example, GenBank Accession Numbers NM 000116.3, NM_181311.2, NM_181312.2, and NMJ81313.2.

The prevalence of Barth Syndrome is estimated at 1/454,000 live births, with an estimated incidence ranging from 1/400,000 to 1/140,000 depending on geographic location. Barth Syndrome is an X-linked disorder, and so disproportionately affects male patients.

The mutations in the TAZ gene comprised in a subject suffering from Barth syndrome result in a decreased taffazin expression level and/or the expression of a defective taffazin.

In the context of this application, a normal taffazin expression level is defined as the expression level of taffazin in a healthy subject, preferably measured in a muscle tissue or a muscle cell. A decreased taffazin expression level means an expression level lower than a normal taffazin expression level, preferably decreased by a factor equal to or lower than 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01.

In the context of this application, a defective taffazin is a taffazin whose catalytic activity to reacylate monolysocardiolipin is decreased, preferably decreased by a factor equal to or lower than 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01 relative to a taffazin expressed in a healthy subject, or no longer comprises such catalytic activity.

Cardiolipin remodeling is compromised in a subject suffering from Barth syndrome, due to a decreased expression of taffazin and/or the expression of a defective taffazin. This can be understood as taffazin catalyzes monolysocardiolipin during cardiolipin remodeling. Without being bound to this theory, Barth syndrome is characterized by an accumulation of monolysocardiolipin. Said accumulation of monolysocardiolipin may be toxic and/or may disrupt or decrease, partially or entirely, the function of cardiolipins comprised in the inner mitochondrial membrane as described above. Inter alia, said accumulation of monolysocardiolipin may disrupt or decrease, partially or entirely, cardiolipins (i) serving as a proton trap, (ii) regulating, facilitating and/or participating in the physical structure of the membrane and complexes comprised therein and (iii) playing a role in apoptosis.

In specific aspects, an inhibitor according to the invention is for use in the treatment and/or prevention of Barth syndrome.

In specific aspects is provided an inhibitor of an ABHD18 protein according to the invention, wherein said ABHD18 protein is expressed in an subject suffering from Barth syndrome, or wherein said ABHD18 protein is expressed in a corresponding organelle, cell fraction, cell, tissue or organ. Preferably, said inhibitor is for use in the treatment and/or prevention of Barth syndrome.

As explained above, a corresponding composition, membrane, organelle, cell fraction, cell, tissue or organ in the context of Barth syndrome means a composition, membrane, organelle, cell fraction, cell, tissue or organ derived from, isolated from, or serving as in vitro or in vivo model for a subject suffering from Barth syndrome. Preferably, a corresponding organelle, cell fraction, cell, tissue or organ in the context of Barth syndrome is, respectively, an organelle, cell fraction, cell, tissue or organ comprising a mutation in a TAZ gene.

In specific aspects is provided an inhibitor of an ABHD18 protein according to the invention, wherein said ABHD18 protein is expressed in an subject comprising a mutation in a TAZ gene, or wherein said ABHD18 protein is expressed in an organelle, a cell fraction, a cell, tissue or organ comprising a mutation in a TAZ gene. Preferably, said mutation leads to a decreased taffazin expression level and/or the expression of a defective taffazin. More preferably, said inhibitor is for use in the treatment and/or prevention of Barth syndrome.

In specific aspects, an inhibitor of the invention is able to induce one or more of the following changes when said inhibitor is introduced in a subject suffering from Barth syndrome, or in a corresponding composition, membrane, organelle, cell fraction, cell, tissue or organ:
a restored or partially restored average fatty acid residue length, preferably wherein the average fatty acid residue length is increased after introduction of the inhibitor, more preferably wherein the average fatty acid residue length is increased by a factor equal to or greater than 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, or 50; and/or
a restored or partially restored cardiolipin dissociation, preferably wherein the cardiolipin dissociation is decreased after introduction of the inhibitor, more preferably by a factor equal to or lower than 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01; and/or
a restored or partially restored MLCL/CL ratio, preferably wherein the MLCL/CL ratio is decreased after introduction of the inhibitor, more preferably by a factor equal to or lower than 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01, most preferably wherein said MLCL/CL ratio is an MLCL/nCL ratio or an MLCL/mCL ratio; and/or
a restored or partially restored cardiolipin concentration, preferably wherein the cardiolipin concentration is increased after introduction of the inhibitor, more preferably by a factor equal to or greater than 2, 2.5, 3, 3.5, 4, 4.5, 5, 5.5, 6, 6.5, 7, 7.5, 8, 8.5, 9, 9.5 10, 15, 20, 25, 30, 35, 40, 45, 50, 55, 60, 65, 70, 75, 80, 85, 90, 95, 100; and/or
an altered nascent cardiolipin concentration, preferably an altered 70:4, 70:5, 70:6, 68:2, 68:3 and/or 68:4 cardiolipin concentration, preferably wherein the concentration is increased after introduction of the inhibitor, more preferably by a factor equal to or greater than 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5 10, 15, 20, 25, 30, 35, 40, 45, 50, 55, 60, 65, 70, 75, 80, 85, 90, 95, 100; and/or
a restored or partially restored cardiolipin oxidation, preferably wherein the cardiolipin oxidation is decreased after introduction of the inhibitor, more preferably by a factor equal to or lower than 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01; and/or
a restored or partially restored structure and/or function of an inner mitochondrial membrane comprised in said subject, composition, membrane, organelle, cell fraction, cell, tissue or organ; and/or
a restored or partially restored formation and/or structure and/or function of a mitochondrial electron transport chain supercomplexes comprised in said subject, composition, membrane, organelle, cell fraction, cell, tissue or organ; and/or
a restored or partially restored cardiolipin composition; and/or
a restored or partially restored average fatty acid residue desaturation,
wherein a partially restored parameter means that the alteration of said parameter relative to a corresponding composition, organelle, cell fraction, cell, membrane, tissue or organ derived from a healthy subject or to a corresponding healthy subject is significantly smaller after introduction of said inhibitor,
wherein a restored parameter means that said parameter is essentially the same as in a corresponding composition, organelle, cell fraction, cell, membrane, tissue or organ derived from a healthy subject or as in a corresponding healthy subject,
preferably wherein said tissue is a muscle tissue or wherein said cell is a muscle cell,
preferably wherein said inhibitor is for use in the treatment and/or prevention of Barth syndrome.

In particular, such a parameter can be considered “partially restored” when said parameter, following after treatment with the inhibitor in accordance with the invention, is closer in value to the same parameter in a subject not suffering from Barth syndrome, compared to value of the same parameter prior to treatment with said inhibitor, and/or more generally is considered by a skilled person (and in particular, by the treating physician) as an improvement in said parameter.

In specific aspects, Barth syndrome is characterized in a subject by a MLCL/CL ratio, preferably an MLCL/mCL ratio, more preferably a 52:2/72:8 MLCL/CL ratio, higher than or equal to 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10, more preferably wherein said MLCL/CL ratio is determined in a muscle tissue or a muscle cell. Accordingly, a corresponding composition, membrane, organelle, cell fraction, cell, tissue or organ in the context of Barth syndrome may be defined as a composition, membrane, organelle, cell fraction, cell, tissue or organ, respectively, wherein the MLCL/CL ratio is higher than or equal to 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10. More preferably, subjects, organelles, cell fractions, cells, tissues and organs according to these specific aspects comprise a mutation in a TAZ gene, wherein said mutations leads to said MLCL/CL ratio.

In specific aspects, Barth syndrome is characterized in a human subject by a 52:2/72:8 MLCL/CL ratio higher than or equal to 0.3, as determined by a Bloodspot assay.

In specific aspects, an inhibitor of the invention is able to induce one or more of the following changes when said inhibitor is introduced in an organelle, a cell fraction, a cell, tissue, organ or subject comprising a mutation in a TAZ gene, wherein said mutation leads to a MLCL/CL ratio, preferably an MLCL/mCL ratio, more preferably a 52:2/72:8 MLCL/CL ratio, higher than 0.05 in said organelle, cell fraction, cell, tissue, organ or subject:
a restored or partially restored average fatty acid residue length, preferably wherein the average fatty acid residue length is increased after introduction of the inhibitor, more preferably wherein the average fatty acid residue length is increased by a factor equal to or greater than 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, or 50; and/or
a restored or partially restored cardiolipin dissociation, preferably wherein the cardiolipin dissociation is decreased after introduction of the inhibitor, more preferably by a factor equal to or lower than 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01; and/or
a restored or partially restored MLCL/CL ratio, preferably wherein the MLCL/CL ratio is decreased after introduction of the inhibitor, more preferably by a factor equal to or lower than 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01, most preferably wherein said MLCL/CL ratio is an MLCL/nCL ratio or an MLCL/mCL ratio; and/or
a restored or partially restored cardiolipin concentration, preferably wherein the cardiolipin concentration is increased after introduction of the inhibitor, more preferably by a factor equal to or greater than 2, 2.5, 3, 3.5, 4, 4.5, 5, 5.5, 6, 6.5, 7, 7.5, 8, 8.5, 9, 9.5 10, 15, 20, 25, 30, 35, 40, 45, 50, 55, 60, 65, 70, 75, 80, 85, 90, 95, 100; and/or an altered nascent cardiolipin concentration, preferably an altered 70:4, 70:5, 70:6, 68:2, 68:3 and/or 68:4 cardiolipin concentration, preferably wherein the concentration is increased after introduction of the inhibitor, more preferably by a factor equal to or greater than 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5 10, 15, 20, 25, 30, 35, 40, 45, 50, 55, 60, 65, 70, 75, 80, 85, 90, 95, 100; and/or
a restored or partially restored cardiolipin oxidation, preferably wherein the cardiolipin oxidation is decreased after introduction of the inhibitor, more preferably by a factor equal to or lower than 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01; and/or
a restored or partially restored structure and/or function of an inner mitochondrial membrane comprised in said subject, composition, membrane, organelle, cell fraction, cell, tissue or organ; and/or
a restored or partially restored formation and/or structure and/or function of a mitochondrial electron transport chain supercomplexes comprised in said subject, composition, membrane, organelle, cell fraction, cell, tissue or organ; and/or
a restored or partially restored cardiolipin composition; and/or
a restored or partially restored average fatty acid residue desaturation,
wherein said alterations, increases and decreases are relative to the corresponding parameter as measured under essentially the same conditions but without the presence of (e.g. before introduction of) said inhibitor according to the invention. Again, said parameter(s) may be measured in the manner described herein (preferred), or alternatively in a manner known per se which will be clear to the skilled person based on the disclosure herein.

Wherein a restored parameter means that said parameter is essentially the same as in a corresponding composition, organelle, cell fraction, cell, membrane, tissue or organ derived from a healthy subject or as in a corresponding healthy subject,
preferably wherein said tissue is a muscle tissue or wherein said cell is a muscle cell,
preferably wherein said inhibitor is for use in the treatment and/or prevention of Barth syndrome.

Again, in this context, a parameter can in particular be considered “partially restored” when said parameter, following after treatment with the inhibitor in accordance with the invention, is closer in value to the same parameter in a subject not suffering from Barth syndrome, compared to value of the same parameter prior to treatment with said inhibitor, and/or more generally is considered by a skilled person (and in particular, by the treating physician) as an improvement in said parameter.

As explained above, the clinical manifestation of Barth syndrome may be due to the accumulation of monolysocardiolipins. These compounds are formed during cardiolipin remodeling from nascent cardiolipin, wherein this reaction may be catalysed by a phospholipase identified as ABHD18 in the current invention. Therefore, the administration of an inhibitor of an ABHD18 protein could decrease the concentration of accumulated monolysocardiolipins in a subject suffering from Barth syndrome, and increasing the concentration of (nascent) cardiolipin (i.e. decreased the MLCL/CL ratio). Without being bound to this theory, said decreased MLCL/CL ratio could be correlated with a decreased toxicity of accumulated monolysocardiolipins and may restore or partially restore the function of cardiolipins comprised in the inner mitochondrial membrane as described above. Although the increase of cardiolipin in this context is an increase of nascent cardiolipin, such an increase may at least partially fulfill the function of mature cardiolipin in the inner mitochondrial membrane.

Clinical manifestation of Barth syndrome is highly variable. Most subjects develop dilated cardiomyopathy during the first decade of life, and typically during the first year of life, which may be accompanied by endocardial fibroelastosis and/or left ventricular noncompaction. The manifestations of Barth syndrome may begin in utero, causing cardiac failure, foetal hydrops and miscarriage or stillbirth during the 2^{nd}/3^{rd} trimester of pregnancy. Ventricular arrhythmia, especially during adolescence, can lead to sudden cardiac death. There is a significant risk of stroke. Skeletal (mostly proximal) myopathy causes delayed motor milestones, hypotonia, severe lethargy or exercise intolerance. There is a tendency to hypoglycaemia during the neonatal period. Ninety percent of patients show mild to severe intermittent or persistent neutropenia with a risk of septicaemia, severe bacterial sepsis, mouth ulcers and painful gums. Lactic acidosis and mild anaemia may occur. Affected boys usually show delayed puberty and growth delay that is observed until the late teens or early twenties, when a substantial growth spurt often occurs. Patients may also present severe difficulties with adequate food intake. Episodic diarrhoea is common. Many patients have a similar facial appearance with chubby cheeks, deep-set eyes and prominent ears.

Subjects suffering from Barth syndrome or decreased taffazin expression levels can be identified by any or a combination of diagnostic or prognostic assays known in the art. For example, typical symptoms of Barth syndrome include symptoms such as, e.g., cardiomyopathy, skeletal muscle abnormalities, neutropenia, slow development, weak muscle tone, increased levels of organic acids in the urine and blood, and/or frequent bacterial infections, such as pneumonia. In some aspects, the subject may exhibit reduced levels of taffazin expression compared to a normal subject, which is measurable using techniques known in the art. In some aspects, the subject may exhibit one or more mutations in the TAZ gene associated with Barth syndrome, which are detectable using techniques known in the art.

In specific aspects, an inhibitor according to the invention is for use in the treatment and/or prevention of Barth syndrome, and/or for use in the treatment and/or prevention of one or more of the followings symptoms of, or diseases or disorders associated with Barth syndrome:
a cardiac disease or disorder, preferably dilated cardiomyopathy, endocardial fibroelastosis, left ventricular noncompaction and/or heart failure; and/or
a muscle disease or disorder, preferably weak muscle tone or skeletal myopathy, more preferably proximal skeletal proximal myopathy; and/or
hypoglycaemia; and/or
immune diseases or disorders and related infections, preferably neutropenia, septicaemia or frequent bacterial infections; and/or
slow development; and/or
increased levels of organic acids in the urine and blood.

In specific aspects, an inhibitor of the invention is able to induce one or more of the following changes when said inhibitor is introduced in a subject suffering from Barth syndrome:
an increase in distance walked during the 6-minute walk test (6MWT); and/or
an increase in muscle strength as measured by handheld dynamometry; and/or
a decrease in the time determined during a five times sit-to-stand test; and/or
a change in 2-D and 3-D echocardiographic measurements; and/or
a change in in accelerometry counts; and/or
a change in in SWAY application balance assessments; and/or
a change in in Patient Reported Outcomes; and/or
a change in in Clinician Global Impression; and/or
a change in change in biomarkers; and/or
a decrease of treatment related adverse events; and/or
a change in peak oxygen consumption; and/or
an increase of cardiac output; and/or
a change in muscle oxygen extraction; and/or
an increase in exercise tolerance; and/or
an increase in in muscle strength; and/or
an increase in quality of life; and/or
a change in left ventricular systolic strain.

Preferably wherein said inhibitor is for use in the treatment and/or prevention of Barth syndrome.

### Other Disorders

In the context of this application, a mitochondrial disorder is a disease or a disorder caused by, related to and/or characterized by a dysfunctional mitochondrion. A mitochondrial disorder is preferably a mitochondrial respiratory disorder, wherein said mitochondrial respiratory disorder is a disease or a disorder caused by, related to and/or characterized by a dysfunctional mitochondrial electron transport chain. In this context, Barth syndrome may be called a mitochondrial respiratory disorder.

Antiphospholipid syndrome (APS) is an autoimmune disorder characterized by venous and or arterial thrombosis and/or pregnancy morbidity. A requirement for the diagnosis of APS is the presence of antiphospholipid antibodies detected as anticardiolipin antibodies.

Diabetic cardiomyopathy (DCM) is a diabetes-associated structural and functional myocardial dysfunction not related to other confounding traditional factors such as coronary artery disease, hypertension, congenital heart disease or valvular heart disease. Studies on patients harts and animal models have correlated DCM with alterations in cardiolipin content and composition.

Kidney disorder induced by obesity and high fat diets are linked with mitochondrial dysfunction and changes in cardiolipin profile. Peroxidation and degradation of cardiolipin have been shown to play a role in the pathogenesis of several forms of kidney disorders. It has also been shown in mice that a long-term high fat diet causes mitochondrial dysfunction and structural alteration, which may be due to degradation and/or peroxidation of cardiolipin.

Immunological disorders, such as allergy, asthma, autoimmune diseases, auto-inflammatory syndromes and immunological deficiency syndromes, cancer, Parkinson's disease, ischemia reperfusion injury, heart failure, traumatic brain injury and ageing have been linked with mitochondrial dysfunction and changes in cardiolipin profile.

In specific aspects, an inhibitor according to the invention is is for use in the treatment and/or prevention of a mitochondrial disorder, preferably a mitochondrial respiratory disorder, antiphospholipid syndrome, diabetic cardiomyopathy, kidney disorder, preferably kidney disorder induced by obesity and/or high fat diets, an immunological disorder, cancer, Parkinson's disease, ischemia reperfusion injury, heart failure, traumatic brain injury or ageing.

In specific aspects, an inhibitor of the invention is able to induce one or more of the following changes when introduced in a subject suffering from a mitochondrial disorder, preferably a mitochondrial respiratory disorder, antiphospholipid syndrome, diabetic cardiomyopathy, kidney disorder, preferably kidney disorder induced by obesity and/or high fat diets, an immunological disorder, cancer, Parkinson's disease, ischemia reperfusion injury, heart failure, traumatic brain injury or ageing, or in a corresponding composition, membrane, organelle, cell fraction, cell, tissue or organ:
a restored or partially restored average fatty acid residue length, preferably wherein the average fatty acid residue length is increased after introduction of the inhibitor; and/or
a restored or partially restored cardiolipin dissociation, preferably wherein the cardiolipin dissociation is decreased after introduction of the inhibitor, more preferably by a factor equal to or lower than 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01; and/or
a restored or partially restored MLCL/CL ratio, preferably wherein the MLCL/CL ratio is decreased after introduction of the inhibitor, more preferably by a factor equal to or lower than 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01, most preferably wherein said MLCL/CL ratio is an MLCL/nCL ratio or an MLCL/mCL ratio; and/or
a restored or partially restored cardiolipin concentration, preferably wherein the cardiolipin concentration is increased after introduction of the inhibitor, more preferably by a factor equal to or greater than 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5 10, 15, 20, 25, 30, 35, 40, 45, 50, 55, 60, 65, 70, 75, 80, 85, 90, 95, 100; and/or an altered nascent cardiolipin concentration, preferably an altered 70:4, 70:5, 70:6, 68:2, 68:3 and/or 68:4 cardiolipin concentration, preferably wherein the concentration is increased after introduction of the inhibitor, more preferably by a factor equal to or greater than 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5 10, 15, 20, 25, 30, 35, 40, 45, 50, 55, 60, 65, 70, 75, 80, 85, 90, 95, 100; and/or
a restored or partially restored cardiolipin oxidation, preferably wherein the cardiolipin oxidation is decreased after introduction of the inhibitor, more preferably by a factor equal to or lower than 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01; and/or
a restored or partially restored structure and/or function of an inner mitochondrial membrane comprised in said subject, composition, membrane, organelle, cell fraction, cell, tissue or organ; and/or
a restored or partially restored formation and/or structure and/or function of a mitochondrial electron transport chain supercomplexes comprised in said subject, composition, membrane, organelle, cell fraction, cell, tissue or organ; and/or
a restored or partially restored cardiolipin composition; and/or
a restored or partially restored average fatty acid residue desaturation;
wherein a partially restored parameter means that the alteration of said parameter relative to a corresponding composition, organelle, cell fraction, cell, membrane, tissue or organ derived from a healthy subject or to a corresponding healthy subject is significantly smaller after introduction of said inhibitor,
wherein a restored parameter means that said parameter is essentially the same as in a corresponding composition, organelle, cell fraction, cell, membrane, tissue or organ derived from a healthy subject or as in a corresponding healthy subject,
preferably wherein said inhibitor is for use in the treatment and/or prevention of a mitochondrial disorder, preferably a mitochondrial respiratory disorder, antiphospholipid syndrome, diabetic cardiomyopathy, kidney disorder, preferably kidney disorder induced by obesity and/or high fat diets, an immunological disorder, cancer, Parkinson's disease, ischemia reperfusion injury, heart failure, traumatic brain injury or ageing.

### Compositions

In a further aspect, the invention provides a composition comprising an inhibitor according to the invention and a pharmaceutically acceptable excipient. Such compositions are referred to in the current application as compositions according to or of the invention.

In specific aspects, an composition according to the invention is for use as a medicament, preferably for use in the treatment and/or prevention of a disease disclosed herein, more preferably for use in the treatment and/or prevention of a cardiolipin disorder, most preferably for use in the treatment and/or prevention of Barth syndrome.

All specific aspects disclosed above for an inhibitor according to the invention may be applied accordingly for an inhibitor according to the invention comprised in a composition according to the invention.

A composition according to the invention may be presented or formulated as capsules, tablets, powders, granules, solutions, suspensions in aqueous or non-aqueous liquids, edible, oil-in-water liquid emulsions, water-in-oil liquid emulsions, solution, syrups and elixirs, in microencapsulated form, liposome delivery systems, such as small unilamellar vesicles, large unilamellar vesicles and multilamellar vesicles, transdermal patches, ointments, creams, suspensions, lotions, powders, solutions, pastes, gels, drops, sprays, aerosols, oils, lozenges, pastilles, mouth washes, suppositories, enemas, aqueous and non-aqueous sterile injection solutions, and so on. It will be appreciated that the compositions may include other agents conventional in the art having regard to the type of formulation.

Non-limiting examples of a pharmaceutically acceptable carrier comprised in a composition are saline, solvents, dispersion media, coatings, antibacterial and antifungal agents, isotonic and absorption delaying agents, and the like, compatible with pharmaceutical administration. Supplementary active compounds, besides an inhibitor according to the invention, can also be incorporated into the compositions.

A composition according to the invention formulated as solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

A composition according to the invention formulated as compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

In a composition according to the invention prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

A composition according to the invention formulated as oral compositions generally include an inert diluent or an edible carrier. For the purpose of oral therapeutic administration, the inhibitor according to the invention can be incorporated with excipients and used in the form of tablets, troches, or capsules, e.g., gelatin capsules. Oral compositions can also be prepared using a fluid carrier for use as a mouthwash.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

A composition according to the invention may be formulated for administration by inhalation, the inhibitor according to the invention can be delivered in the form of an aerosol spray from a pressurized container or dispenser, which contains a suitable propellant, e.g., a gas such as carbon dioxide, or a nebulizer.

A composition according to the invention may be formulated for transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one aspect, transdermal administration may be performed my iontophoresis.

A composition according to the invention may comprise a carrier system such as a colloidal system. The colloidal system can be a liposome, a phospholipid bilayer vehicle. In one aspect, the inhibitor according to the invention is encapsulated in a liposome. An inhibitor according to the invention can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

### Therapeutic Uses

In specific aspects, an inhibitor according to the invention for use as a medicament or a composition according to the invention is for use as a medicament, preferably for use in the treatment and/or prevention of a disease disclosed herein, more preferably for use in the treatment and/or prevention of a cardiolipin disorder, a cardiolipin remodeling disorder, Barth syndrome, antiphospholipid syndrome, diabetic cardiomyopathy or kidney disorder induced by obesity and/or high fat diets.

All specific aspects disclosed above for an inhibitor according to the invention may be applied accordingly for an inhibitor according to the invention for such use as a medicament and for a composition according to the invention for such use as a medicament.

Wherever an inhibitor according to the invention for use as a medicament or a composition according to the invention is for use as a medicament is disclosed in this application, a corresponding method for the manufacture or the production of a medicament comprising such an inhibitor or such a composition is also disclosed. Accordingly, the use of such an inhibitor or such a composition as a medicament is also envisioned.

A medicament according to the invention is an inhibitor according to the invention for use as a medicament or a composition according to the invention is for use as a medicament, preferably for use in the treatment and/or prevention of a disease disclosed herein.

A medicament according to the invention may be administered orally, nasally, buccally, sublingually, vaginally, parenterally, topically, systemically, intravenously, subcutaneously, intraperitoneally, intramuscularly, intrathecally, by inhalation or epidurally.

A medicament according to the invention, preferably for use in the treatment and/or prevention of Barth syndrome, may be co-administered separately, sequentially or simultaneously with a cardiovascular agent to a subject in need thereof. In some aspects, the cardiovascular agent is selected from the group consisting of: an anti-arrhythmia agent, a vasodilator, an anti-anginal agent, a corticosteroid, a cardioglycoside, a diuretic, a sedative, an angiotensin converting enzyme (ACE) inhibitor, an angiotensin II antagonist, a thrombolytic agent, a calcium channel blocker, a throboxane receptor antagonist, a radical scavenger, an anti-platelet drug, a β-adrenaline receptor blocking drug, a-receptor blocking drug, a sympathetic nerve inhibitor, a digitalis formulation, an inotrope, and an antihyperlipidemic drug.

A medicament according to the invention, preferably for use in the treatment and/or prevention of Barth syndrome, may be co-administered separately, sequentially or simultaneously with antibiotics, granulocyte colony stimulating factor (GCSF), and agents for the control of cardiac conditions, including but not limited to, for example, diuretics, ACE inhibitors, digoxin (digitalis), calcium channel blockers, and beta-blockers. In mild cases, thiazide diuretics, such as hydrochlorothiazide at 25-50 mg/day or chlorothiazide at 250-500 mg/day, are useful. However, supplemental potassium chloride may be needed, since chronic diuresis causes hypokalemis alkalosis. Moreover, thiazide diuretics usually are not effective in patients with advanced symptoms of Barth syndrome. Typical doses of ACE inhibitors include captopril at 25-50 mg/day and quinapril at 10 mg/day.

A medicament according to the invention, preferably for use in the treatment and/or prevention of Barth syndrome, may be co-administered separately, sequentially or simultaneously with an adrenergic beta-2 agonist. An “adrenergic beta-2 agonist” refers to adrenergic beta-2 agonists and analogues and derivatives thereof, including, for example, natural or synthetic functional variants, which have adrenergic beta-2 agonist biological activity, as well as fragments of an adrenergic beta-2 agonist having adrenergic beta-2 agonist biological activity. The term “adrenergic beta-2 agonist biological activity” refers to activity that mimics the effects of adrenaline and noradrenaline in a subject and which improves myocardial contractility in a patient having Barth Syndrome. Commonly known adrenergic beta-2 agonists include, but are not limited to, clenbuterol, albuterol, formeoterol, levalbuterol, metaproterenol, pirbuterol, salmeterol, and terbutaline.

A medicament according to the invention, preferably for use in the treatment and/or prevention of Barth syndrome, may be co-administered separately, sequentially or simultaneously with an adrenergic beta-1 antagonist. Adrenergic beta-1 antagonists and adrenergic beta-1 blockers refer to adrenergic beta-1 antagonists and analogues and derivatives thereof, including, for example, natural or synthetic functional variants which have adrenergic beta-1 antagonist biological activity, as well as fragments of an adrenergic beta-1 antagonist having adrenergic beta-1 antagonist biological activity. Adrenergic beta-1 antagonist biological activity refers to activity that blocks the effects of adrenaline on beta receptors. Commonly known adrenergic beta-1 antagonists include, but are not limited to, acebutolol, atenolol, betaxolol, bisoprolol, esmolol, and metoprolol.

As used herein, the term “simultaneous” therapeutic use refers to the administration of at least two active ingredients by the same route and at the same time or at substantially the same time. The term “separate” therapeutic use refers to an administration of at least two active ingredients at the same time or at substantially the same time by different routes. The term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

In the context of this application, the terms “preventing”, “prevention”, treating” or “treatment”, respectively refer to therapeutic treatment, wherein the object is to prevent, reduce, alleviate or slow down (lessen), respectively and as applicable, the targeted pathologic disorder or disease and/or its progression in a subject. In particular, said terms relate to a treatment which has the object of improving one or more symptoms and/or physiological parameters that are caused by, associated with and/or characteristic of the disease or disorder that is to be treated, and/or the object to preventing that such symptom(s) to arise and/or that such symptom(s) or physiological parameter(s) further deteriorate. Based on his general knowledge and the further disclosure herein, the skilled person (and in particular, the treating physician) will be able to suitably determine and measure said symptom(s) or physiological parameter(s), depending on the specific disease involved.

For example, a subject is successfully “treated” for Barth syndrome if, after receiving a therapeutic amount of an inhibitor or a composition according to the invention, the subject shows observable and/or measurable reduction in or absence of one or more signs and symptoms of Barth syndrome, such as, e.g., cardiomyopathy, skeletal muscle abnormalities, neutropenia, slow development, weak muscle tone, increased levels of organic acids in the urine and blood, and/or frequent bacterial infections, such as pneumonia. It is also to be appreciated that the various modes of treatment or prevention of medical conditions as described are intended to mean “substantial,” which includes total but also less than total treatment or prevention, and wherein some biologically or medically relevant result is achieved.

In the context of this application, the terms “prevention” or “preventing” of a disorder or disease refers to a compound that, in a statistical sample, reduces the occurrence of symptoms of a disorder or disease in the treated sample relative to an untreated control sample, or delays the onset or reduces the severity of one or more symptoms of the disorder or condition relative to the untreated control sample. For example, preventing Barth syndrome includes preventing or delaying the initiation of, preventing, delaying, or slowing the progression or advancement of, and/or reversing the progression of Barth syndrome. As used herein, prevention of a disorder or disease, such as Barth syndrome, also includes preventing a recurrence of one or more signs or symptoms said disorder or disease.

A medicament according to the invention is administered to a subject in need thereof in an effective amount (i.e., amount that have desired therapeutic effect). Preferably, an effective amount refers to an amount of an inhibitor according to the invention comprised in said medicament. The dose and dosage regimen will depend upon the degree of the infection in the subject, the characteristics of the particular inhibitor according to the invention, e.g., its therapeutic index, the subject, and the subject's history. Certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the medicaments according to the invention can include a single treatment or a series of treatments.

The effective amount may be determined during pre-clinical trials and clinical trials by methods familiar to physicians and clinicians. An effective amount of a peptide useful in the methods may be administered to a subject in need thereof by any of a number of well-known methods for administering pharmaceutical compounds.

Dosage, toxicity and therapeutic efficacy of a medicament according to the invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Medicaments that exhibit high therapeutic indices are preferred.

The data obtained from the cell culture assays and animal studies can be used in formulating a range of dosage for use in humans. The dosage lies preferably within a range of circulating concentrations that include the ED50 with little or no toxicity. The dosage may vary within this range depending upon the dosage form employed and the route of administration utilized. For any medicament according to the invention, the therapeutically effective dose can be estimated initially from cell culture assays.

### Double Mutants

In a further aspect, the invention provides an organelle, a cell fraction, a cell, tissue, organ or subject comprising a mutation in a TAZ gene and a mutation in an ABHD18 gene. An organelle, a cell fraction, a cell, tissue, organ or subject according to this aspect may be called a double mutant according to the invention.

In specific aspects, a double mutant according to the invention is obtainable by combining an organelle, a cell fraction, a cell, tissue, organ or subject comprising a mutation in a TAZ gene with an organelle, a cell fraction, a cell, tissue, organ or subject comprising a mutation in an ABHD18 gene. More preferably, said combining is a hybridisation.

In specific aspects, said mutations in said TAZ and ABHD18 genes result in a decreased expression level of or an expression of a defective taffazin and ABHD18 protein, respectively.

A decreased expression level means an expression level lower than a normal expression level, preferably decreased by a factor equal to or lower than 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01, wherein a normal expression level is defined as the expression level of in a corresponding organelle, cell fraction, cell, tissue, organ or subject not comprising a corresponding mutation.

In the context of this application, a defective taffazin or ABHD18 protein is a taffazin or ABHD18 protein whose catalytic activity is decreased, preferably decreased by a factor equal to or lower than 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01 relative to a taffazin or ABHD18 protein expressed in a corresponding organelle, cell fraction, cell, tissue, organ or subject not comprising a corresponding mutation, or no longer comprises such catalytic activity.

In specific aspects, the MCL/CL ratio in a double mutant according to the invention is equal to or lower than 2.5, 2, 1.5, 1, 0.9, 0.8, 0.7, 0.6, 0.5, 0.4, 0.3, 0.2, 0.1, 0.095, 0.09, 0.085, 0.08, 0.075, 0.07, 0.065, 0.06, 0.055, 0.05. More preferably, said MLCL/CL ratio is an MLCL/nCL or an MLCL/mCL ratio.

In specific aspects, a double mutant according to the invention is a non-human animal, more preferably a non-human vertebrate, most preferably a non-human mammal.

### Assays

In an aspect, the invention provides a method for screening or identifying an inhibitor according to the invention.

In specific aspects, the invention provides a method for screening or identifying an inhibitor according to the invention comprising a competitive assay. Preferably, the competitive assay comprises the steps of (a) mixing two types of cells, preferably in a 1:1 ratio, wherein the types of cells differ in the level and/or the type of taffazin and/or ABHD18 expressed, and (b) incubating part of the mixed cells in the presence of a compound and incubating another part of the mixed cells in the absence of the compound, (c) monitoring one or more cell viability parameters of each type of cells during both incubations. Suitable examples of cell viability parameters are, without being limiting, the number of cells (e.g. competitive growth assay), the number of cell deaths (e.g. TUNEL assay) or metabolic activity (e.g. XTT assay, Resazurin assay). Suitable types of cells may be selected, without being limiting, from wild type, taffazin knock-out (TAZ KO), ABHD18 knockout (ABHD18 KO) or (taffazin+ABHD18) double knockout (DKO) cells, wherein in each of these cells wild-type or mutant ABHD18 may be overexpressed, wherein the two types of cells should differ as described above, preferably wherein the cells are Hap-1 cells.

In more specific aspects, the invention provides a method for screening or identifying an inhibitor according to the invention comprising a competitive growth assay (i.e. a type of competitive assay as described above). Preferably, the competitive growth assay comprises the steps of (a) mixing two types of cells in a 1:1 ratio, wherein the types of cells differ in the level and/or the type of taffazin and/or ABHD18 expressed, and (b) incubating part of the mixed cells in the presence of a compound and incubating another part of the mixed cells in the absence of the compound, (c) monitoring the number of each type of cells during both incubations. Wherever reference is made to a competitive assay in this application, this type of assay is meant, unless explicitly mentioned otherwise. Suitable types of cells may be selected, without being limiting, from wild type, taffazin knock-out (TAZ KO), ABHD18 knockout (ABHD18 KO) or (taffazin+ABHD18) double knockout (DKO) cells, wherein in each of these cells wild-type or mutant ABHD18 may be overexpressed, wherein the two types of cells should differ as described above, preferably wherein the cells are Hap-1 cells. The number of cells of the types of cells in the incubations may be monitored (i.e. measured) via fluorescence-activated cell sorting (FACS). The difference between the incubation with and the incubation without the compound in the ratio of the number of cells of each type, preferably 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 days after the start of the incubations, is related to the suitability of the compound as an inhibitor for use as described herein. This difference can easily be interpreted by the skilled person in view of the clinical mechanism described herein.

As a specific example, and without being limiting, the competitive growth assay may comprise the steps of (a) mixing TAZ knock-out (KO) Hap-1 cells with ABHD18-TAZ double knock-out (DKO) Hap-1 cells in a 1:1 ratio; (b) incubating part of the mixed cells in the presence of a compound and incubating another part of the mixed cells in the absence of the compound, (c) monitoring the number of KO and DKO cells during both incubations. The difference between the incubation with and the incubation without the compound in the ratio of the number of DKO cells to the number of KO cells positively correlates with the suitability of the compound as an inhibitor for use as described herein.

In alternative aspects, the invention provides a method for screening or identifying an inhibitor according to the invention comprising a competitive activity-based protein profiling (ABPP). This technique has been widely used to screen for or identify inhibitors for various ABHD proteins, as described in Bononi, Giulia, et al. “α/β-Hydrolase Domain (ABHD) Inhibitors as New Potential Therapeutic Options against Lipid-Related Diseases.” Journal of medicinal chemistry 64.14 (2021): 9759-9785.

In alternative aspects, the invention provides a method for screening or identifying an inhibitor according to the invention comprising a p-nitrophenylbutyrate (pNPB) assay. This technique is commonly used to screen for inhibitors of serine hydrolases, as described in Iglesias, Jose, et al. “Simplified assays of lipolysis enzymes for drug discovery and specificity assessment of known inhibitors.” Journal of lipid research 57.1 (2016): 131-141.

### Definitions

All documents cited in the present specification are hereby incorporated by reference in their entirety. Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

Unless indicated otherwise, all methods, steps, techniques and manipulations that are not specifically described in detail can be performed and have been performed in a manner known per se, as will be clear to the skilled person. Reference is made to the standard handbooks, to the general background art referred to above and to the further references cited therein.

As used herein, the singular forms ‘a’, ‘an’, and ‘the’ include both singular and plural referents unless the context clearly dictates otherwise.

The terms ‘comprising’, ‘comprises’ and ‘comprised of’ as used herein are synonymous with ‘including’, ‘includes’ or ‘containing’, ‘contains’, and are inclusive or open-ended and do not exclude additional, non-recited members, elements or method steps.

The recitation of numerical ranges by endpoints includes all numbers and fractions subsumed within the respective ranges, as well as the recited endpoints.

Physiological conditions are defined in the context of this application as typical environmental conditions in a vertebrate, mammalian or human cell or tissue that is in homeostasis and is not subject to extraordinary external stress. Preferably, physiological conditions mean a temperature from 25° C. up to 45° C., more preferably from 30° C. up to 40° C.

A fatty acid residue is defined in the context of this application as a residue of an essentially linear carboxylic acid after esterification, preferably wherein the number of carbon atoms comprised in said residue is from 8 up to 30, more preferably from 10 up to 28, most preferably from 12 up to 24. In this context, an essentially linear carboxylic acid preferably means an unbranched alkyl chain terminally attached to a carboxylic acid chain, wherein the combined, total number of carbon atoms comprised in said unbranched alkyl chain and said carboxylic is from 8 up to 30, more preferably from 10 up to 28, most preferably from 12 up to 24 carbon atoms. Optionally, said unbranched alkyl chain may be substituted with one or more functional groups and/or one or more additional alkyl chains, wherein the total number of carbon atoms comprised in each of said additional alkyl chains is 1, 2, 3 or 4.

A CX: Y fatty acid residue is defined in the context of this application as a fatty acid residue, wherein the number of carbon atoms comprised in said fatty acid residue is X and wherein the number of carbon-carbon formal double bonds comprised in said fatty acid residue is Y.

A concentration is preferably a molar concentration, preferably a molar concentration per weight or per volume, most preferably measured under physiological conditions.

A subject is defined in the context of this application as a (living) organism, unless explicitly stated otherwise. A subject may be any organism, including invertebrates and vertebrates. Preferably, a subject is a vertebrate. More preferably, a vertebrate is a starfish or a mammal. Even more preferably, a mammal is a rat, a mouse, a rabbit or a human. Most preferably, a mammal is a human. In an alternative specific aspect, a subject is a non-human animal, more preferably a non-human vertebrate, most preferably a non-human mammal.

An organ is preferably a muscle. A tissue is preferably a muscle tissue, more preferably a cardiac muscle tissue, a skeletal muscle tissue or a smooth muscle tissue, most preferably a cardiac muscle tissue or a skeletal muscle tissue. A cell is preferably a muscle cell, more preferably a cardiac muscle cell, a skeletal muscle tissue or a smooth muscle cell, most preferably a cardiac muscle cell or a skeletal muscle cell. An organelle is preferably an mitochondrion. The organ, tissue or cell is preferably of, derived from, isolated from, or serving as in vitro or in vivo model for a subject as defined above, more preferably wherein said subject is suffering from a disorder or a disease as specified herein, most preferably wherein said disorder or disease is Barth syndrome.

An increase of a parameter by a factor equal to or higher than X is defined in the context of this application as a change of said parameter from its initial value A to a value equal to or higher than A*X. For example, the increase of a MLCL/CL ratio of 1.0 by a factor equal to or higher than 3.0 means the increase of said MLCL/CL ratio to a value equal to or higher than 3.0.

An increase of a parameter by a factor equal to or lower than X is defined in the context of this application as a change of said parameter from its initial value A to a value equal to or lower than A*X. For example, the increase of a MLCL/CL ratio of 1.0 by a factor equal to or lower than 3.0 means the increase of said MLCL/CL ratio to a value higher than 1.0 but equal to or lower than 3.0.

A decrease of a parameter by a factor equal to or lower than X is defined in the context of this application as a change of said parameter from its initial value A to a value equal to or lower than A*X. For example, the decrease of a MLCL/CL ratio of 2.0 by a factor equal to or lower than 0.1 means the decrease of said MLCL/CL ratio to a value equal to or lower than 0.2.

A decrease of a parameter by a factor equal to or higher than X is defined in the context of this application as a change of said parameter from its initial value A to a value equal to or higher than A*X. For example, the decrease of a MLCL/CL ratio of 2.0 by a factor equal to or higher than 0.1 means the decrease of said MLCL/CL ratio to a value lower than 2.0 but equal to or higher than 0.2.

A parameter that is essentially the same as in a corresponding composition, organelle, cell fraction, cell, membrane, tissue or organ derived from a healthy subject or as in a corresponding healthy subject, preferably means that the value of said parameter cannot be distinguished by a skilled person from the value of a corresponding parameter in a corresponding composition, organelle, cell fraction, cell, membrane, tissue or organ derived from a healthy subject or in a corresponding healthy subject, and/or that the value of said parameter would be interpreted by a skilled person as measured in a corresponding composition, organelle, cell fraction, cell, membrane, tissue or organ derived from a healthy subject or in a corresponding healthy subject.

An alteration of a parameter which is significantly smaller after introduction of an inhibitor in a composition, organelle, cell fraction, cell, membrane, tissue, organ or subject preferably means that the absolute difference between the value of said parameter and the value of a corresponding parameter in a corresponding composition, organelle, cell fraction, cell, membrane, tissue or organ derived from a healthy subject or in a corresponding healthy subject is decreased by a factor equal to or lower than 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02 or 0.01 after said introduction.

Any parameter referred to herein is preferably determined using the specific method, assay or methodology described herein. Where the present specification does not mention or describe a specific method, assay or methodology for determining said parameter, said parameter can be measured in a manner suitable per se, as will be clear to the skilled person based upon reading the present disclosure.

A small molecule is defined in the context of this application as a term commonly used in molecular biology and pharmacology for referring to an organic compound having a low molecular weight (<900 Daltons) with a size on the order of 1 nm. Because of their upper molecular-weight limit of 900 Daltons, small molecules can rapidly diffuse across cell membranes to reach intracellular sites of action (e.g. Golgi). Preferably, a small molecule has a molecular weight lower than 500 Daltons.

Each amino acid sequence described herein by virtue of its identity or similarity percentage (at least 60%) with a given amino acid sequence respectively has in a further specific aspect an identity or a similarity of at least 60%, 65%, 70%, 75%, 80%, 85%, 90%, 95%, 97%, 98%, 99% or more identity or similarity with the given amino acid sequence respectively. In a specific aspect, sequence identity or similarity is determined by comparing the whole length of the sequences as identified herein. Unless otherwise indicated herein, identity or similarity with a given SEQ ID NO means identity or similarity based on the full length of said sequence (i.e. over its whole length or as a whole).

Sequence identity is defined in the context of this application as a relationship between two or more amino acid (polypeptide or protein) sequences or two or more nucleic acid (polynucleotide) sequences, as determined by comparing the sequences. The identity between two amino acid sequences is preferably defined by assessing their identity within a whole SEQ ID NO as identified herein or part thereof. Part thereof may mean at least 50% of the length of the SEQ ID NO, or at least 60%, or at least 70%, or at least 80%, or at least 90%.

In the art, sequence identity also means the degree of sequence relatedness between amino acid sequences, as the case may be, as determined by the match between strings of such sequences. Sequence similarity between two amino acid sequences is determined by comparing the amino acid sequence and its conserved amino acid substitutes of one polypeptide to the sequence of a second polypeptide. Sequence identity and similarity can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine sequence identity are designed to give the largest match between the sequences tested. Methods to determine sequence identity and similarity are codified in publicly available computer programs. Preferred computer program methods to determine sequence identity and similarity between two sequences include e.g. the GCG program package (Devereux, J., et al., Nucleic Acids Research 12 (1): 387 (1984)), BestFit, FASTA, BLASTN, and BLASTP (Altschul, S. F. et al., J. Mol. Biol. 215:403-410 (1990)), EMBOSS Needle (Madeira, F., et al., Nucleic Acids Research 47 (W1): W636-W641 (2019)). The BLAST program is publicly available from NCBI and other sources (BLAST Manual, Altschul, S., et al., NCBI NLM NIH Bethesda, MD 20894; Altschul, S., et al., J. Mol. Biol. 215:403-410 (1990)). The EMOSS program is publicly available from EMBL-EBI. The well-known Smith Waterman algorithm may also be used to determine identity. The EMBOSS Needle program is the preferred program used.

Preferred parameters for polypeptide sequence comparison include the following: Algorithm: Needleman and Wunsch, J. Mol. Biol. 48 (3): 443-453 (1970); Comparison matrix: BLOSUM62 from Henikoff and Henikoff, Proc. Natl. Acad. Sci. USA. 89:10915-10919 (1992); Gap Open Penalty: 10; and Gap Extend Penalty: 0.5. A program useful with these parameters is publicly available as the EMBOSS Needle program from EMBL-EBI. The aforementioned parameters are the default parameters for a Global Pairwise Sequence alignment of proteins (along with no penalty for end gaps).

Preferred parameters for nucleic acid comparison include the following: Algorithm: Needleman and Wunsch, J. Mol. Biol. 48:443-453 (1970); Comparison matrix: DNAfull; Gap Open Penalty: 10; Gap Extend Penalty: 0.5. A program useful with these parameters is publicly available as the EMBOSS Needle program from EMBL-EBI. The aforementioned parameters are the default parameters for a Global Pairwise Sequence alignment of nucleotide sequences (along with no penalty for end gaps).

Optionally, in determining the degree of amino acid (sequence) similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; a group of amino acids having acidic side chains is aspartate and glutamate; and a group of amino acids having sulphur-containing side chains is cysteine and methionine. Preferred conservative substitutions for each of the naturally occurring amino acids are as follows: Ala to Ser; Arg to Lys or Gln; Asn to Asp, His or Ser; Asp to Glu or Asn; Gln to Glu, Lys or Arg; Glu to Lys, Asp, Gln; His to Tyr or Asn; Ile to Leu, Val, or Met; Leu to Ile, Met or Val; Lys to Arg, Gln or Glu; Met to Val, Leu or Ile; Phe to Trp or Tyr; Ser to Thr, Ala or Asn; Thr to Ser; Trp to Tyr or Phe; Tyr to His, Trp or Phe; and Val to Ile, Leu or Met. Substitutional variants of the amino acid sequence disclosed herein are those in which at least one residue in the disclosed sequences has been removed and a different residue inserted in its place. Preferably, the amino acid change is conservative.

### LEGEND TO THE FIGURES

FIG. 1—ABHD18 identified as a common modifier in both Cell-Seq and synthetic viability map. A) Cell-Seq map generated using antibody against mtCOX1 in HAP1 wild type (WT) and TAZ Ko cells. Mutations in TAZ appear to enhance mtCOXI levels in WT. ABHD18 appears to restore mtCOXI levels to an extent in TAZ Ko cells. B) Two viability-based maps generated in independent clonal TAZ Ko cell lines. In both datasets ABHD18 and ADIPOR2 loss-of-function appear to enhance fitness of cells deficient for TAZ. C) Cell-Seq compendium reveals that besides mtCOXI, ABHD18 loss affects the levels of the lipid LBPA (lysobisphosphatidic acid) and no other currently mapped cellular phenotypes.

FIG. 2—Biosynthesis and remodeling of cardiolipin in yeast and human. Phosphatidic acid (PA) is converted to CDP-diacylglycerol (CDP-DAG) by CDP-DAG synthase. Phosphatidylglycerolphosphate synthase catalyzes the conversion of CDP-DAG to phosphatidylglycerolphosphate (PGP), which is dephosphorylated to phosphatidylglycerol (PG). PG is converted to nascent CL with mostly saturated acyl chains. Nascent CL is deacylated to monolyso-CL (MLCL) by Cld1 in yeast (human orthologue is not known). MLCL is reacylated to mature CL with mostly unsaturated acyl chains by TAZ.

FIG. 3—ABHD18 loss rescues deficient mitochondrial supercomplex assembly in TAZ Ko HAP1 cells. BN-PAGE immunoblot analysis of supercomplexes formed by complexes I+III+IV in digitonin-solubilized mitochondria using antibody against complex IV subunit (mtCOX-1) on wild-type (WT), TAZ Ko, ABHD18 Ko and TAZ-ABHD18 double Ko HAP1 cells. TAZ Ko cells showed destabilization of higher order supercomplexes containing multiple copies of complex IV (upper panel). A faster migration of the monomeric complex IV was also observed in TAZ Ko cells (middle panel). Loss of ABHD18 in TAZ Ko cells rescues higher order supercomplex assembly along with normal migration of the monomeric complex IV.

FIG. 4—ABHD18 loss rescues the growth defect of TAZ KO Hap-1 cells in a competitive growth assay. ABHD18-TAZ KO cells were mixed with either TAZ KO cells or WT-Hap-1 cells in 1:1 ratio on day 1 and percentage of individual cell populations was measured by FACS over a period of 35 days. ABHD18-TAZ DKO shows a clear growth advantage over TAZ KO cells and almost similar growth compared to WT Hap-1 cells.

FIG. 5—ABHD18 deletion rescues MLCL accumulation in TAZ Ko cells and creates a shifts towards CL species with saturated acyl chains. TAZ Ko cells exhibit accumulation of total MLCL (A), decrease in total CL (B) and increased total MLCL/total CL ratio (C). Loss of ABHD18 in TAZ Ko cells rescues total MLCL accumulation and shifts MLCL/CL ratio back to wild-type (WT) cells. D) Bubble plots represent the abundance and diversity of CL subspecies present in different cell types, where x-axis indicates number of total carbons in fatty acyl chains while y-axis indicates number of double bonds in fatty acyl chains. Bubble size represents relative average abundance of each subspecies, normalized to protein and comparable between four genotypes. TAZ Ko cells show a decrease in unsaturated acyl chains (no of double bonds) compared to WT cells. ABHD18 loss in WT and TAZ KO cells changes cardiolipin composition, namely an increase in short (carbon chain length) and saturated acyl chains (less no of double bonds) containing CL species. E) Phosphatidylglycerol (PG) and F) Phosphatidic acid (PA) are increased in TAZ Ko cells and loss of ABHD18 rescues PG and PA levels back to WT Hap-1 cells. All the plots represent the values that is average of 4 different WT clones and 3 different clones of each of the other genotypes

FIG. 6—Dox-induced GFP tagged ABHD18 expression in Hap-1 cells shows mitochondrial localization of ABHD18 protein.

FIG. 7—Morphological and functional parameters of Zebrafish larvae and hearts at 5 days post fertilization (dpf) show a pronounced cardiomyopathy in TAZ mutants. Following parameters were measured during cardio phenotyping: A) lateral view of a representative control and taz F0 mutant larvae; B) percentage of embryos showing heart edema; C) maximum diameter of heart's atria D) maximum diameter of heart's ventricle E) ejection fraction; F) heart rate expressed in Beats Per Minute; G) QTc corrected interval. H) Survival analysis show an apparent increased mortality during early development of TAZ mutants (between 20- and 30-dpf). Detailed methodology can be found in Example 15.

FIG. 8—LC-MS spectra at the retention time of cardiolipin of a bloodspot extract of a control wild type mouse (A) and a ABHD18 HOM knock out mouse (B) shows a clear shift towards shorter and more saturated acyl chain containing CL species in ABHD18 HOM mice. Relative intensities in the spectra were normalized against the highest peak, i.e., m/z 1449.98 for the control, m/z 1427.99 for ABHD18 HOM mouse. 40 μl blood from each mice was spotted per spot on a Guthrie card and cardiolipin spectra was measured by HPLC-MS/MS. 3 wild type and 3 ABHD18 KO mice were taken for the study.

FIG. 9—The growth defect in TAZ KO cells, is reconstituted in TAZ/ABHD18 DKO cells by expressing wildtype ABHD18[WT], but not catalytic dead ABHD18[S199A] mutant. Co-cultures of HAP1 Cherry and HAP1-parent, HAP1-TAZ KO, HAP1-ABHD18 KO, or HAP1-TAZ/ABHD18 DKO cell clones expressing ABHD18[WT]-EGFP or ABHD18[S199A]-EGFP were followed over time by flow cytometry in a competitive growth assay. The EGFP+ proportion is plotted against the time from the start of co-culture (Day 0). The bar-graphs show the average EGFP+ proportion (mean+/−SD; n=3) of single cell clones after expression of ABHD18[S199A] compared to ABHD18[WT].

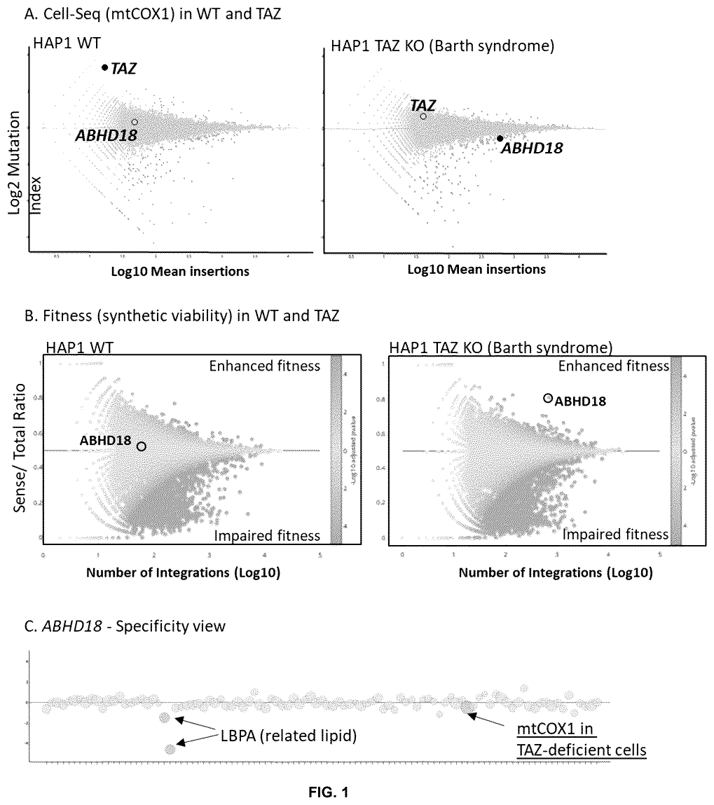

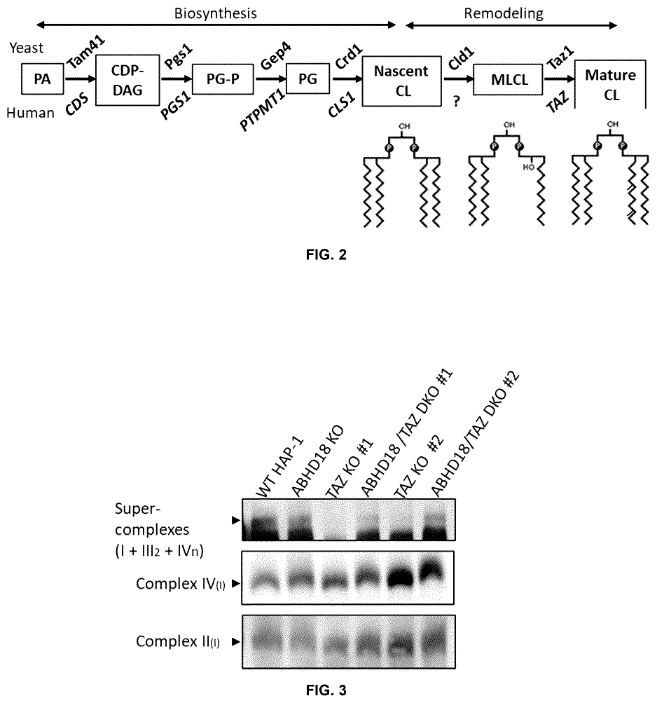

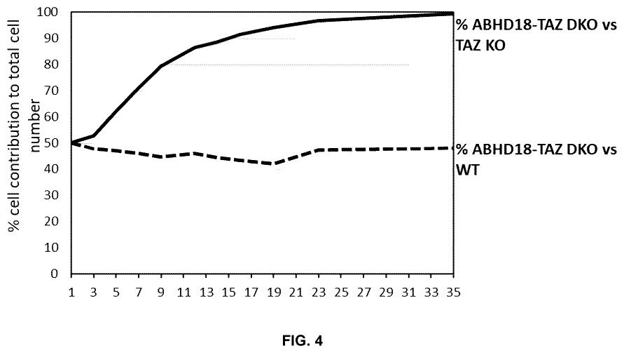

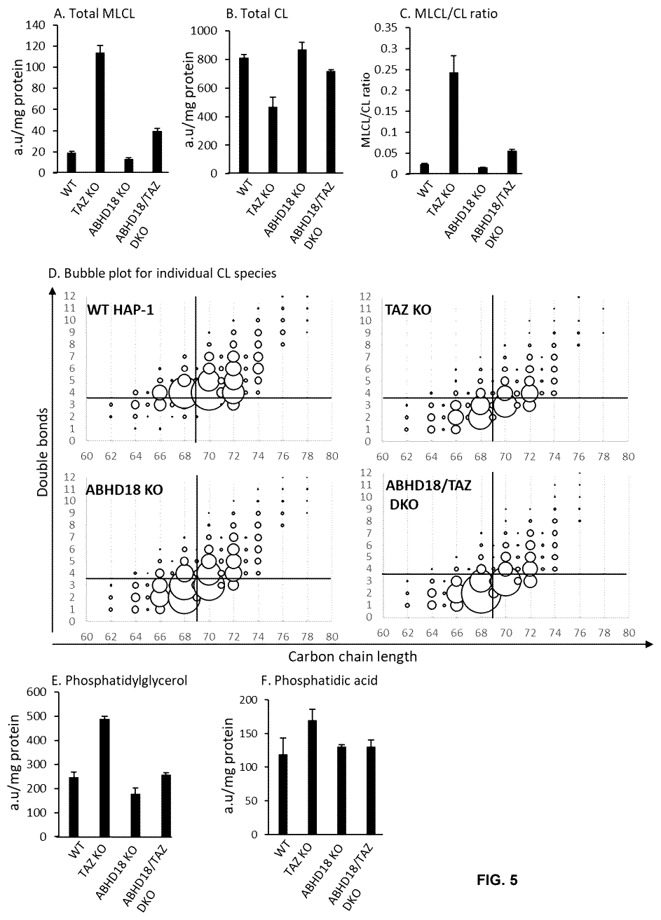

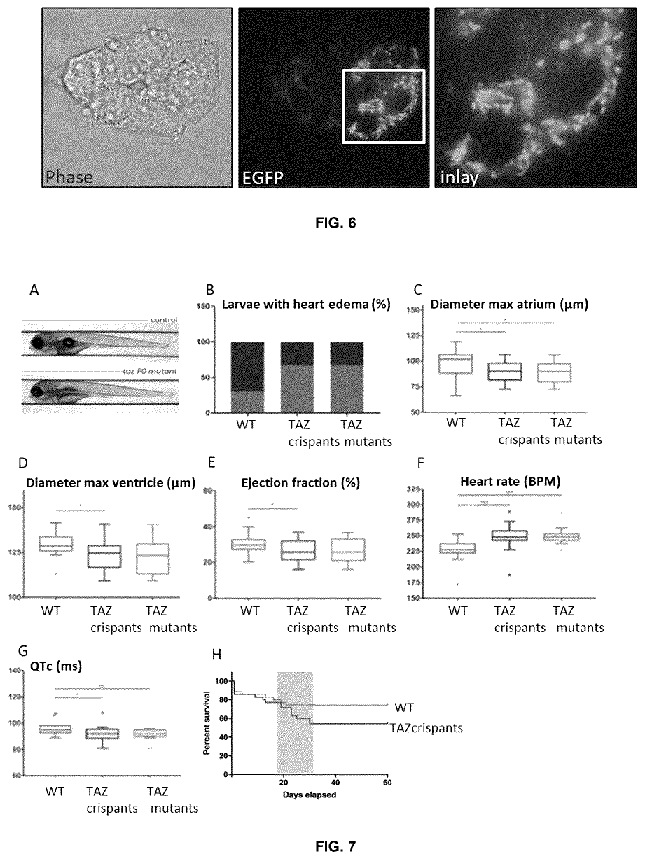

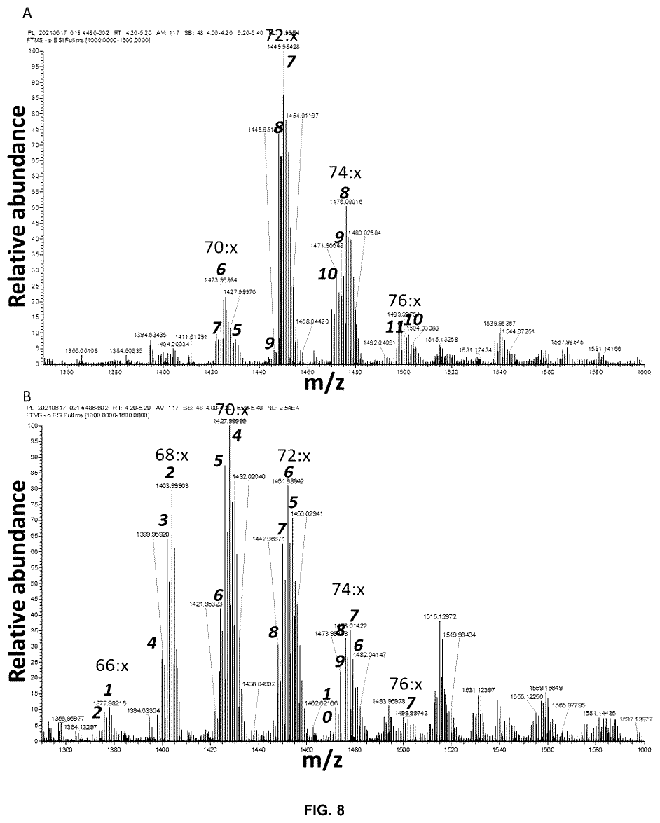

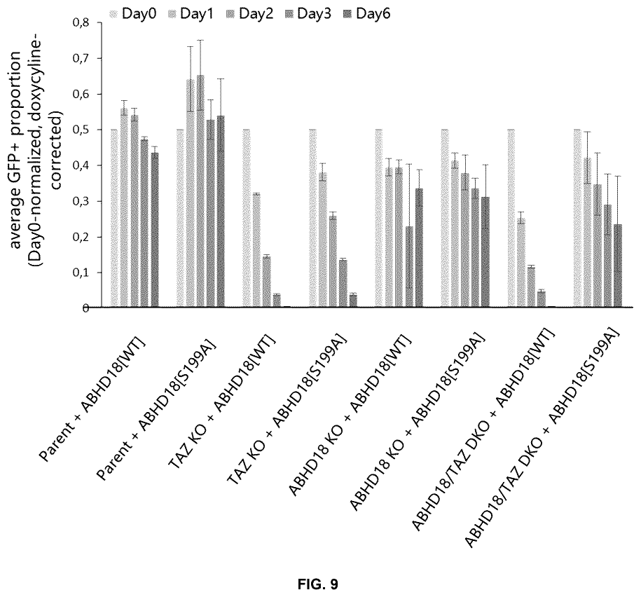

### EXAMPLES

The following examples are offered for illustrative purposes only, and are not intended to limit the scope of the present invention in any way.

### Example 1: Cell Lines

HAP1 cells (Carette et al., 2011, Nature, Vol. 477, pages 340-343) were cultured in Iscove's modified Dulbecco's medium (IMDM, ThermoFisher Scientific) supplemented with heat-inactivated 10% fetal calf serum (FCS, VWR), 100 U/ml penicillin, 100 μg/ml streptomycin and 2.92 μg/ml L-glutamine solution (ThermoFisher Scientific). HEK293T were obtained from authentic stocks (ATCC) and maintained in Dulbecco's modified Eagle's medium (DMEM, ThermoFisher Scientific) containing the above-mentioned supplements.

### Example 2: Generation of Vectors

LentiCRISPRv2 vector (Genscript) was cut with PacI and EcoRI restriction enzymes (New England Biolabs) and the backbone was isolated from gel using a Qiaquick Gel Extraction Kit (Qiagen) according to the manufacturer's protocol. A synthetic DNA fragment (CAGGGACAGCAGAGATCCAGTTTGGTTAATTAAGGTACCGAGGGCCTATTTCCCATGAT TCCTTCATATTTGCATATACGATACAAGGCTGTTAGAGAGATAATTAGAATTAATTTGACT GTAAACACAAAGATATTAGTACAAAATACGTGACGTAGAAAGTAATAATTTCTTGGGTAGT TTGCAGTTTTAAAATTATGTTTTAAAATGGACTATCATATGCTTACCGTAACTTGAAAGTAT TTCGATTTCTTGGCTTTATATATCTTGTGGAAAGGACGAAACACCGGAGACGGATTAATT AAACCGTCTCAGTTTAAGAGCTAGAAATAGCAAGTTTAAATAAGGCTAGTCCGTTATCAA CTTGAAAAAGTGGCACCGAGTCGGTGCTTTTTTGAATTCGCTAGCTAGGTCTTGAAAGGA GTGG, IDTDNA, SEQ ID NO: 7) was inserted using NEBuilder HiFi DNA Assembly Master Mix (New England Biolabs) to obtain pSCNC-LentiCRISPR. pSCN-LentiCRISPR-eGFP was generated by cutting the puromycin resistance cassette of pSCNC-LentiCRISPR with PacI and EcoRI restriction enzymes (New England Biolabs) and replacing with eGFP cassette.

### Example 3: Generation of HAP-1 Knockout Cell Line

Knockout cell lines were generated by using the CRISPR/Cas9 system. sgRNAs targeting TAZ (sgTAZ #1_exon 2 (SEQ ID NO: 8): 5′-CCTGACCGTGCACAACAGGG-3′ and sgTAZ #2_exon 2 (SEQ ID NO: 9): 5′-CCTGACCGTGCACAACAGGG-3′) and ABHD18 (sgABHD18 #1_exon 4 (SEQ ID NO: 10): 5′-CAATTGAATCTGTTATTGCA-3′ and sgABHD18 #2_exon 8 (SEQ ID NO: 11): 5′-ATGACTGGAATATCCATGGG-3′) were cloned into pSCNC-LentiCRISPR. BsmBI (New England Biolabs) digested pSCNC-LentiCRISPR was isolated from gel using a Qiaquick Gel Extraction Kit (Qiagen) and oligonucleotides (SEQ ID NO: 12: 5′TATCTTGTGGAAAGGACGAAACACCGNNNNNNNNNNNNNNNNNNNNGTTTAAGAGCTA GAAATAGCAAGTTTAAA-3′, where N is the sgRNA) encoding sgRNAs targeting TAZ or ABHD18 were cloned in using NEBuilder HiFi DNA Assembly Master Mix (New England Biolabs) to obtain pSCNC-LentiCRISPR-TAZ and pSCNC-LentiCRISPR-ABHD18 respectively. HAP1 cells were transfected with pSCNC-LentiCRISPR-TAZ or pSCNC-LentiCRISPR-ABHD18 using Lipofectamin 2000 (ThermoFisher Scientific) according to the manufacturer's instructions. After 24 hours incubation, cells were selected with puromycin (Invivogen, 1 μg/ml) for 48 hours. Resistant colonies were clonally expanded, and single clones were verified by PCR and Sanger sequencing (primers listed in the table below). To create double knockouts, TAZ-knockout cells were transfected with two pSCNC-LentiCRISPR-ABHD18.

| | | SEQ |
|---|---|---|
| Primer name | Sequence (5′-3′) | ID NO |
| Fwd_sgTAZ#1 | CACCTACAGCTGCTTCTGGAC | 13 |
| Rev_sgTAZ#1 | GCTGTAAGATATGCCCTGTGG | 14 |
| Fwd_sgTAZ#2 | CACCTACAGCTGCTTCTGGAC | 15 |
| Rev_sgTAZ#2 | GCTGTAAGATATGCCCTGTGG | 16 |
| Fwd_sgABHD18#1 | AACATCCATGACAGACCAAGA | 17 |
| Rev_sgABHD18#1 | TGCAACTATAAACAATGCTTCAA | 18 |
| Fwd_sgABHD18#2 | GAGGCTAGAAGGTCCAGCTTA | 19 |
| Rev_sgABHD18#2 | GTAACAGCAAAATTGGGCATT | 20 |

### Example 4: Generation of EGFP Tagged ABHD18 Expressing Inducible Landing Platform HAP-1 Cells

PIN510A-1 vector (Sanbio PinPoint HR kit) was modified to generate Tet-ON inducible expression system. EGFP tagged ABHD18 construct was cloned in the Tet-ON inducible expression system to obtain PINTRE-ABHD18[WT]-EGFP. HAP-1 cells harboring the PinPoint attP site at AAVS1 locus were co-transfected with PINTRE-ABHD18[WT]-EGFP and PIN200A-1 (Sanbio) using Lipofectamin 2000 (ThermoFisher Scientific). After 48 hours incubation, cells were selected with blasticidin (Invivogen, 20 μg/ml). To induce expression, cells were treated with 2 μg/ml doxycycline (Sigma) for 48 hours at 37° C.

### Example 5: Blue Native PAGE and Western Blotting

Mitochondrial supercomplex assembly was studied by BN-PAGE, as previously described (Jha et al., 2017; doi: 10.1002/9780470942390.mo150182). Briefly, mitochondria were isolated using Abcam mitochondrial isolation kit (ab110170) and isolated mitochondrial pellet (125 μg of protein) was solubilized for 20 min on ice in 20 μl of sample buffer cocktail containing 5% digitonin (Novex, cat. No. BN2006, digitonin to protein ratio was kept to 4 g/g) and sample buffer (4×; Novex, cat. No. BN20032). Insoluble material was removed by centrifugation at 20,000 g for 10 min at 4° C. and the soluble component was combined with Coomassie G-250 (final G-250 concentration was ¼^{th} of the digitonin concentration). Samples were separated on a 3-12% gradient gel (NativePAGE, Novex, cat. No. BN2011BX10) using Xcell SureLock Mini-Cell system (Novex, cat. No. EI0001). For separation, the inner chamber was first filled with dark blue cathode buffer (5% 20× NativePAGE™ cathode buffer additive-Novex, cat. No. BN2002 and 5% 20× NativePAGE Running Buffer-Novex, cat. No. BN2001) until the dye front had reached approximately one-third of the gel. Then the dark cathode buffer was exchange with light blue cathode buffer (0.5% 20× NativePAGE™ cathode buffer additive-Novex, cat. No. BN2002 and 5% 20× NativePAGE Running Buffer-Novex, cat. No. BN2001). The running buffer used was NativePAGE Running Buffer (20×; Novex, cat. No. BN2001). Native complexes were separated at 150 V for 30 min and then switched to 250 V for 180 min. Following electrophoresis, BN-PAGE gel was incubated in transfer buffer (NuPAGE Transfer Buffer (20×; Novex, cat. No. NP0006-1) for 15 min. Resolved proteins were transferred to PVDF membrane using Western blotting (90 min at 150 mA). Membrane was washed with 8% acetic acid to fix the proteins before destaining first with 100% methanol. For immunodetection, PVDF membrane was blocked overnight with 2.5% (w/v) skim milk powder in TRIS buffered saline containing 0.1% (v/v) Tween-20. For the immunodetection of protein complexes, we used monoclonal antibodies (Abcam) against the following subunits: 70 kDa subunit of complex II, 37 kDa subunit of complex IV.

### Example 6: Competitive Growth Assay

sgRNA against ABHD18 was cloned into pSCNC-LentiCRISPR or pSCN-LentiCRISPR-eGFP vectors to generate pSCNC-LentiCRISPR-ABHD18 and pSCN-LentiCRISPR-eGFP-ABHD18. For lentivirus production, HEK293T cells were co-transfected with pSCNC-LentiCRISPR-ABHD18, pSCN-LentiCRISPR-eGFP-ABHD18 or empty vector controls and the packaging plasmid mix (pCgpV, VSVg and pRSV-REV) using TurboFectin 8.0 (Origene). Viral supernatant was harvested 48 hours post transfection and subsequently filtered (0.45 μm), concentrated by centrifugation using Amicon® Ultra-15 Centrifugal Filter Units at 4000 rpm for 20 min and stored at −80° C.

HAP-1 WT and two different TAZ Ko clones were transduced with concentrated virus and were selected using puromycin or FACS-sorted based on GFP expression. Once selected/sorted cells were expanded, four different combinations of GFP positive and negative cells were generated by mixing them in equal numbers (1 million of each cell line).
1. TAZ KO (pSCNC-LentiCRISPR-ABHD18)+WT (pSCN-LentiCRISPR-eGFP)
2. TAZ KO (pSCNC-LentiCRISPR-eGFP-ABHD18)+WT (pSCN-LentiCRISPR)
3. TAZ KO (pSCNC-LentiCRISPR-ABHD18)+TAZ KO (pSCN-LentiCRISPR-eGFP)
4. TAZ KO (pSCNC-LentiCRISPR-eGFP-ABHD18)+TAZ KO (pSCN-LentiCRISPR)

The day of mixing was marked as day 1 (8 days after virus infection). Proliferation of these cell lines was tested by following the percentage of GFP positive and negative cells by FACS over a period of 35 days.

### Example 7: Haploid Genetic Screens

Retroviral genome-wide mutagenesis of haploid HAP1 wild-type and TAZ KO cells was performed as previously described (Brockmann et al., 2017, doi: 10.1038/nature22376). In short, gene trap retrovirus was produced in HEK293T cells by co-transfection of the packaging plasmids Gag-pol, VSVg, and pAdvantage, gene trap plasmid and blue fluorescent protein using TurboFectin 8.0 (Origene). Viral supernatant was harvested after 48 hours following transfection and subsequently filtered (0.45 μm) and concentrated by centrifugation using Amicon® Ultra-15 Centrifugal Filter Units at 4000 rpm for 20 min. Concentrated virus was stored overnight at 4° C. and this procedure was repeated after 24 hours. To mutagenize HAP1 WT or TAZ KO cells, 25 million haploid cells were seeded in a 15 cm dish and transduced with concentrated gene trap retrovirus 24 h after plating. The mutant library was expanded for 7 days following the infection with the mutagen, frozen and used for genetic screens. 3 billion expanded mutagenized cells were fixed, permeabilized and stained and sorted into two separate populations based on the fluorescent intensity of the MTCO-1 (Abcam) antibody staining BD FACSAria III as described previously (Brockmann et al., 2017, doi: 10.1038/nature22376). 10 million cells were sorted for low and high populations each.

The gene trap insertion sites were amplified using a LAM-PCR procedure as previously described (Brockmann et al., 2017, doi: 10.1038/nature22376). Using a biotinylated primer in the gene trap cassette, single-stranded DNA (ssDNA) products were generated for 120 cycles, captured on magnetic beads, a pre-adenylated ssDNA linker ligated to the 3′ end, and a final round of exponential amplification using a primer containing Illumina sequencing compatible overhangs at the end of the LTR and in the ssDNA linker. Following PCR purification (QIAGEN) samples were sequenced on a NextSeq (Illumina).

| | | SEQ |
|---|---|---|
| Primer name | Sequence | ID NO |
| 5′Dual-biotin-LAM-PCR | /52-Bio/GGTCTCCAAATCTCGGTGGAAC | 21 |
| P7_TG | CAAGCAGAAGACGGCATACGA | 22 |
| MultiplexLinkerBC01_TG | /5Phos/AGATCGGAAGAGCACACGTCTGAACTCCAGTCAC | 23 |
| | ATTACTCGATCTCGTATGCCGTCTTCTGCTTG/3ddC/ | |
| MultiplexLinkerBC02_TG | /5Phos/AGATCGGAAGAGCACACGTCTGAACTCCAGTCAC | 24 |
| | TCCGGAGAATCTCGTATGCCGTCTTCTGCTTG/3ddC/ | |
| MultiplexLinkerBC03_TG | /5Phos/AGATCGGAAGAGCACACGTCTGAACTCCAGTCAC | 25 |
| | CGCTCATTATCTCGTATGCCGTCTTCTGCTTG/3ddC/ | |
| MultiplexLinkerBC04_TG | /5Phos/AGATCGGAAGAGCACACGTCTGAACTCCAGTCAC | 26 |
| | GAGATTCCATCTCGTATGCCGTCTTCTGCTTG/3ddC/ | |
| Illumina | AATGATACGGCGACCACCGAGATCTACACAGGCTATA | 27 |
| MultiplexPCRBC01_TG | GATGGTTCTCTAGCTTGCC | |
| Illumina | AATGATACGGCGACCACCGAGATCTACACGCCTCTAT | 28 |
| MultiplexPCRBC02_TG | GATGGTTCTCTAGCTTGCC | |
| Illumina | AATGATACGGCGACCACCGAGATCTACACAGGATAG | 29 |
| MultiplexPCRBC03_TG | GGATGGTTCTCTAGCTTGCC | |
| Illumina | AATGATACGGCGACCACCGAGATCTACACTCAGAGC | 30 |
| MultiplexPCRBC04_TG | CGATGGTTCTCTAGCTTGCC | |
| SolexaSeqFlank_TG | CTAGCTTGCCAAACCTACAGGTGGGGTCTTTCA | 31 |
| (Sequencing primer) | | |
| MultiplexIndex2Read_TG | CACCTGTAGGTTTGGCAAGCTAGAGAACCATC | 32 |
| (Sequencing primer) | | |

Insertion site mapping and analysis were performed as previously described Brockmann et al., 2017, doi: 10.1038/nature22376. After deep sequencing of the low- and high sorted populations, gene-trap insertion sites were determined as unique reads aligning unambiguously to the human genome (hg19) using bowtie (10.1186/gb-2009-10-3-r25), allowing for a single mismatch. Aligned reads were mapped using hg19 protein-coding gene coordinates (Refseq) to identify intragenic insertion sites and their orientation with respect to the gene using intersectBED (10.1093/bioinformatics/btq033.). For this analysis, insertion sites integrated in sense within a gene were considered disruptive. To prevent potential confounding, insertion sites in genomic regions assigned to overlapping genes were discarded, as well as integrations in the 3′ untranslated region (UTR) of genes as the gene-trap cassette might have been less effective there in ablating gene function. To identify genes enriched for disruptive gene-trap integrations in either the high- or low-query populations, the number of unique disruptive mutations in each gene and in the total of one population (for example, signal high) was counted and compared with those values in the other population (for example, signal low) using a two-sided Fisher's exact test. Resulting P values were adjusted for multiple testing using the Benjamini-Hochberg false discovery rate correction. For each gene, a mutation index (MI) was calculated corresponding to the ratio of the number of disruptive integrations per gene in both populations normalized by the number of total integrations in each channel. For genes without a single insertion site in only one of the channels, a value of 1 was assigned so as not be omitted from the plots.

### Example 8: Synthetic Viability Screens

Genes required for viability of two independent TAZ KO cell lines were profiled as previously described in detail (Blomen et al., 2015). In short, gene trap retrovirus was produced in HEK293T cells by co-transfection of the packaging plasmids Gag-pol, VSVg, pAdvantage, together with BFP-containing gene trap plasmid using TurboFectin 8.0 (Origene). Viral supernatant was harvested after 48 hours following transfection and subsequently filtered (0.45 μm) and concentrated by centrifugation using Amicon® Ultra-15 Centrifugal Filter Units at 4000 rpm for 20 min. Concentrated virus was stored overnight at 4° C. and this procedure was repeated after 24 hours.

To mutagenize HAP1 WT or TAZ KO cells, 25 million haploid cells were in a 15 cm dish and transduced with concentrated gene trap retrovirus 24 h after plating. The mutagenized TAZ KO cells were passaged for 13 days following the infection with the mutagen, collected after dissociation using trypsin-EDTA by pelleting, and fixed using fix buffer I (BD biosciences). In order to minimize confounding from diploid cells carrying heterozygous mutations, the fixed cells were stained using DAPI for DNA content and sorted for haploid cells in the G1 phase of the cell cycle on a BD FACSAria III. Genomic DNA was isolated from 30 million sorted cells using a DNA mini kit (QIAGEN) with the lysis step occurring overnight at 56° C. for de-crosslinking.

The gene trap insertion sites were amplified using a LAM-PCR procedure as previously described (Brockmann et al., 2016). Using a biotinylated primer in the gene trap cassette, single-stranded DNA (ssDNA) products were generated for 120 cycles, captured on magnetic beads, a pre-adenylated ssDNA linker ligated to the 3′ end, and a final round of exponential amplification using a primer containing Illumina sequencing compatible overhangs at the end of the LTR and in the ssDNA linker. Following PCR purification (QIAGEN) samples were sequenced on a NextSeq (Illumina). Insertion sites were mapped by aligning the deep sequencing reads to the human genome (hg19) using bowtie (Langmead et al., 2009) allowing for a single mismatch. Reads were cropped to 50 bp. Unique aligned reads were subsequently assigned to Refseq gene coordinates using Bedtools (Quinlan and Hall, 2010). Overlapping gene regions on strands were disregarded (since orientation bias in that region is not readily interpretable). For each replicate a binomial test for the distribution of sense and antisense orientation insertions was performed and corrected for multiple testing using a Benjami-Hochberg FDR correction. To identify genetic modifiers that enhance fitness of TAZ-deficient cells, the TAZ KO datasets were normalized to four control datasets (as described in Blomen et al., 2015) and the numbers of sense and antisense integrations in each gene was compared to WT datasets using a Fisher's exact test. Only genes that significantly deviated from all 4 WT datasets (accessible through the NCBI SRA accession number SRP058962) and showed more sense than antisense integrations were considered as candidate genetic suppressors of TAZ-deficiency.

### Example 9: Lipidomics

### Sample Preparation

Haploid HAP-1 cells were cultured in parallel in 15-cm dishes. After they reached confluence, the cells were dissociated using trypsin-EDTA and counted. 50 million cells (˜2.5 mg of protein) for each cell line were pelleted by centrifugation at 1500 rpm for 4 min. The pelleted cells were washed twice with PBS and twice with 0.9% NaCl (Sigma) followed by centrifugation at 4° C. (1500 rpm for 4 min). After final wash, the pellets were completely dried and stored-80 degrees.

### Method

The frozen cell pellet was shipped for lipidomics analysis, which was performed at Core Facility Metabolomics, Amsterdam UMC.

Extraction of phosopholipids and analysis was done as previously described (https://doi.org/10.1194/jlr.M067470).

The HPLC system consisted of an Ultimate 3000 binary HPLC pump, a vacuum degasser, a column temperature controller, and an auto sampler (Thermo Scienti_c, Waltham, MA, USA). The column temperature was maintained at 25° C. The lipid extract was injected onto a “normal phase column “LiChrospher 2×250-mm silica-60 column, 5 μm particle diameter (Merck, Darmstadt, Germany) and a “reverse phase column” Acquity UPLC HSS T3, 1.8 μm particle diameter (Waters, Milford Massachusetts, USA). A Q Exactive Plus Orbitrap (Thermo Scienti_c) mass spectrometer was used in the negative and positive electrospray ionization mode. In both ionization modes, mass spectra of the lipid species were obtained by continuous scanning from m/z 150 to m/z 2000 with a resolution of 280,000 full width at half maximum (FWHM).

### Processing

An in-house developed pipeline, written in the R programming language, was used for data processing. The RAW data files were converted to mzXML using Msconvert (https://doi.org/10.1038/nbt.2377) in Centroided mode. Peak finding and peak group finding were done using the R package XCMS, with minor modifications to some functions for a better representation of the Q Exactive data. Annotation of the peaks was done based on an in-house database containing all possible phospholipid species. Each combination of column (normal phase or reverse phase) and scan mode (positive or negative) was processed separately; after normalization, separate peak group lists were combined into two resulting lists, which were used for statistical analysis.

### Identification

Identification is based on exact mass (with 3 ppm tolerance) and retention time including the relation between these two parameters, taking into account the different molecular species of the lipid class (see: Herzog et al: https://doi.org/10.1194/jlr.M067470). Our identification is thus mostly tentative, the current experiment should be seen as a screen to identify interesting features/species that should be further analyzed to confirm their identity (by MS/MS, NMR or other methods).

### Levels of Confidence

A set of internal standards (ISs), compounds with the same general structure as the lipid classes of interest, has been added to each sample in the dataset after sample work-up and before data collection. These ISs have been adequately identified and are used both to locate all other metabolites in the same chemical class and to normalize the intensities for those metabolites. The ISs have confidence level 1 (see Sumner et al, https://doi.org/10.1007/s11306-007-0082-2) and other metabolites in the same class have confidence level 3. Our approach is semi-quantitative at best and results cannot be directly compared to other measurements that have been done in separate experiments. The reported response of the lipid species (defined as the ratio between the abundance of a selected lipid species and the abundance of the corresponding internal standard) can only be used to compare the same lipid species across different samples/groups.

Other lipid classes, for which no IS was added, are annotated as well. These metabolites have confidence level 4 along with species that have been annotated both in reversed phase and in normal phase. These additional species are annotated based on their m/z values and their observed retention time pattern, which is consistent with what is expected. In the case where no appropriate internal standard is present for a certain class of lipids, their intensities are normalized on the intensity of one IS (usually PE), to correct for inter-batch variation only.

### Example 10: Generation of ABHD18 Knock Out Mice

ABHD18 KO mice were generated using the CRISPR/Cas9 system at Jackson Laboratory. In brief, two sgRNAs (sgRNA_up-ATGGATCAAGTGTGGGGTTG and sgRNA_down-GCAATTAGGATACAGTACCA) were microinjected into fertilized embryos of C57BL/6J mice.

Deletions of >8 kb region that includes exon 8 of Abhd18 were confirmed by Sanger sequencing. Homozygous KO mice were born from a heterozygous intercross. All mice were genotyped using PCR with specific primers (forward (SEQ ID NO: 33), 5′-TCCTCTCTGGAATAAAAAGA-3′ and reverse (SEQ ID NO: 34), 5′-CATCTATAGCAAACTTCACA-3′).

### Example 11: Blood Spot Assay on ABHD18 Knock-Out Mice

The blood spot assay on WT control and ABHD18 KO mice was done at Amsterdam, UMC as previously described (DOI: 10.1373/clinchem.2007.095711). Briefly, 45-50 ul of blood was drawn from three different ABHD18 homozygous mice (age 8-9 weeks, 2 males, 1 female) and three age matched WT control mice (3 females). EDTA-anticoagulated blood was shipped by Jackson Laboratory. 40 ul blood from each mouse were spotted on Guthrie card using 200 ul pipette (obtained from Amsterdam, UMC) and air dried for 6-7 hours. Cardiolipin spectra was measured by HPLC-MS/MS.

### Example 12: Generation of PINTRE-ABHD18[WT]-EGFP (Puromycin) and PINTRE-ABHD18[S199A]-EGFP (Puromycin) Plasmids

The Pinpoint donor vector PIN510A (Sanbio) was modified to contain the doxycycline inducible promoter rtTA[TetON 3G], and a puromycin selection cassette. gBLOCKS containing ABHD18[WT]-EGFP or ABHD18[S199A]-EGFP sequences were cloned into the modified donor vector using EcoRI and AgeI restriction sites.

### Example 13: Generation of EGFP-Tagged ABHD18 Expressing Inducible Landing Platform HAP-1 TAZ KO, ABHD18 KO and TAZ/ABHD18 DKO Cell Clones

Knockout inducible landing platform HAP-1 cell lines were generated using the CRISPR/Cas9 system, and transfection with pSCNC-LentiCRISPR-sgTAZ #1 and pSCNC-LentiCRISPR-sgABHD18 #2, as described. To create double knockouts, pSCNC-LentiCRISPR-sgTAZ #1 and pSCNC-LentiCRISPR-sgABHD18 #2 were co-transfected.

HAP-1 TAZko, ABHD18ko and TAZ/ABHD18 dko cell clones harboring the PinPoint attP site at AAVS1 locus were co-transfected with PINTRE-ABHD18[WT or S199A]-EGFP (puromycin) and PIN200A-1 (Sanbio) using Lipofectamine 2000 (ThermoFisher Scientific). After 48 hours incubation, cells were selected with puromycin (1 μg/mL). To induce ABHD18-GFP expression, cells were treated with 2 μg/mL doxycycline (Sigma) for 24 hours at 37 C.

### Example 14: Competitive Cell Growth Assay with Functional Reconstitution of ABHD18

HAP1 clonal cell lines were counted and 1.5M cells were co-cultured with 1.5M HAP1 Cherry cells in IMDM medium including 10% FBS and antibiotics in 6 well plates. Two wells per condition were seeded. One well was treated with 2 μg/mL doxycycline and the other well left untreated. 24 hours after start of co-culture was labelled as Day 0. On Days 0, 1, 2, 3, and 6, the proportions of EGFP positive (EGFP+) cells were determined using BD FACSAria Fusion or BD FACSAria III. The proportion of EGFP+ cells over time is used as a measure for cell proliferation. The proportion of EGFP+ cells was normalized to the proportion on Day 0 (normalize to 0.5). In the untreated control wells, the Cherry-negative cells represent the clonal cell lines, and their proportions were used to correct for background proliferation. The average EGFP+ proportion of three independent clonal cell lines was calculated. The average relative EGFP+ proportion was calculated as the average EGFP+ proportion in TAZ/ABHD18 DKO cells corrected for the average EGFP+ proportion in TAZ KO cells.

### Example 15: Zebrafish Experiments

### Materials

E3 medium
Tricaine (ETHYL 3-AMINOBENZOATE METHANESULFONATE SALT) (Sigma Aldrich)
CoBoXtract (CoBo Scientific)
TEMPase Hot Start DNA Polymerase (AMPLIQON)
Alt-R® S.p. Cas9 Nuclease V3 (IDT biotechnology)
Alt-R® CRISPR-Cas9 tracrRNA (IDT biotechnology)

### Primers

Primers were purchased from IDT biotechnology (25 nmole DNA oligo)

| Gene | | |
|---|---|---|
| Name | Primer Name | Sequence (5′ to 3′) |
| Taz | Taz.1 fw FAM | AGCTGACCGGCAGCAAAATTGTTTTGCTTGCGCTTTTATCCCA |
| | | (SEQ ID NO: 35) |
| | Taz.2 rev | GCAAGAACCGCATGAACAGTAA |
| | | (SEQ ID NO: 36) |

sgRNAs

sgRNAs were purchased from IDT biotechnology (Alt-R® CRISPR-Cas9 crRNA, 2 nmol)

| Gene Name | crRNA Name | Sequence (5′ to 3′) |
|---|---|---|
| Taz | taz.target1 | CGATGCCCTTGGAGGTGACA |
| | | (SEQ ID NO: 37) |
| | taz.target4 | GATTTCACTTACTGGTCCAA |
| | | (SEQ ID NO: 38) |

### Methods

### Zebrafish Maintenance

Adult wild-type zebrafish (Danio rerio), strain AB, purchased from KIT-European Zebrafish Resource Centre (EZRC)—were maintained at 24-25° C. on a light cycle of 14 h light: 10 h dark (lights on at 7 am; lights off at 9 pm)

### Preparation of Cas9/sgRNA Complexes

Cas9 endonuclease (Alt-R® S.p. Cas9 Nuclease V3) and sgRNAs (crRNA and tracrRNA) were purchased from IDT biotechnologies. Cas9/sgRNA ribonucleoprotein complexes were assembled according to manufacturer's instruction.

### Cas9/sgRNA Microinjections

One-cell-stage embryos were injected with a mix containing the Cas9/sgRNA complexes following published protocols (http://sfvideo.blob.core.windows.net/sitefinity/docs/default-source/usersubmitted-method/crispr-cas9-rnp-delivery-zebrafish-embryosjessnerc46b5a1532796e2eaa53ff00001c1b3c.pdf?sfvrsn=52123407_4).

Evaluation of sgRNAs Efficiency

Upon injection, sgRNAs cutting efficiency was evaluated by the IDAA™ (INDEL detection by amplicon analysis) method (https://coboscientific.com/genome-editing/indel-detection-byamplicon-analysis-idaatm/).

Genomic DNA was extracted in CoboXtract™ (https://coboscientific.com/genomeediting/coboxtracttm-quick-dna-extraction-solution/) buffer from single embryos, following manufacturer's instructions.

The PCR was setup and performed by TEMPase Hot Start DNA Polymerase (AMPLIQON) following the guidelines provided in the kit.

CoBoScientific-Biomedical Research (https://coboscientific.com/) performed the INDEL Profile analysis. INDEL Profiling graphs were obtained by using the analysis software Profilelt™

### Cardiac Activity Characterization

Analysis of Cardiac Function Phenotype: At 5 dpf, tg(myl7:GFP) larvae previously injected with control sgRNA/Cas9 or taz sgRNA/Cas9 were anesthetized and placed in a robotic microfluidic system VAST (Union Biometrica), which allows the automatic aspiration, placement and rotation of the larvae under the microscope (Leica, DM6-B). Once they are positioned under the right angle, videos of the fluorescent heart (myl7:GFP) were recorded during 20 sec. Videos were analyzed by ZeCardio® software for the presence of the following heart dysfunctions:
Bradycardia and Tachycardia*.
QTc corrected interval: Framingham formula: QTc=QT+0.154 (1−RR)
Arrhythmia
Atrial and Ventricular Fibrillation
Cardiac arrest
Ejection Fraction (EJF %)
Premature Ventricular Contraction (PVC) phenotypes: Bigeminy, Trigeminy, PVC run.
*Bradycardia and Tachycardia are calculated in comparison with control larvae.

After video acquisition, larvae were individually genotyped and the results from video analysis through ZeCardio® software were grouped by genotype.

### Statistical Analysis

Data was analyzed using the GraphPad Prism software version 8.0 (Armonk, NY, USA). Data are presented as mean±standard error of the mean (SEM). Statistical analysis of the data for heart rate, ventricular arrhythmia, cardiac arrests, QTc interval and ejection fraction was performed using Welch corrected t-test. Results were statistically compared between taz specific and control sgRNA injected groups. Differences were considered statistically significant when p<0.05 (*<0.05; **: p<0.01; ***: p<0.001).

### Mortality Evaluation

Once they reached the stage of 5 dpf, injected larvae were introduced in the nursery of our 10 fish facility to have optimal growing conditions. The number of alive animals was counted daily. Survival curves were built using the GraphPad Prism software version 8.0 (Armonk, NY, USA).

1. An inhibitor of an ABHD18 protein, wherein said inhibitor is able to decrease an activity of said ABHD18 protein, wherein said activity is an activity of the ABHD18 protein that is involved in cardiolipin biosynthesis and/or metabolism, and in particular in remodeling of a cardiolipin.

2. An inhibitor according to claim 1, wherein said ABHD18 protein is represented by an amino acid sequence having 80% sequence identity with SEQ ID NO: 1.

3. An inhibitor according to claim 1, wherein said ABHD18 protein has a length from 200 up to 480 amino acids.

4. An inhibitor according to claim 1, wherein said inhibitor is able to specifically bind said ABHD18 protein, preferably wherein the binding site comprises at least part of the hydrolase domain of said ABHD18 protein.

5. An inhibitor according to claim 1, wherein said ABHD18 protein is expressed in a cell comprising a mutation in a TAZ gene.

6. An inhibitor according to claim 5, wherein introducing said inhibitor in said cell is able to decrease the concentration ratio of total monolysocardiolipin (MLCL) to total cardiolipin (CL) in said cell.

7. An inhibitor according to claim 5, wherein introducing said inhibitor in said cell is able to restore the formation of a mitochondrial electron transport chain supercomplex comprised in said cell.

8. A composition comprising an inhibitor according to claim 1 and a pharmaceutically acceptable excipient.

9.-13. (canceled)

14. The inhibitor according claim 4, wherein the inhibitor is a small molecule, an antibody fragment, or an aptamer.

15. The inhibitor according claim 5, wherein the mutation leads to a concentration ratio of total monolysocardiolipin (MLCL) to total cardiolipin (CL) higher than 0.1 in said cell.

16. The inhibitor according claim 6, wherein introducing said inhibitor in said cell is able to decrease the concentration ratio of total monolysocardiolipin (MLCL) to total cardiolipin (CL) in said cell by at least 1.5 times.

17. A method for the treatment of a disease or condition associated with cardiolipin biosynthesis, the method comprising administering the inhibitor according to claim 1 to a subject in need thereof.

18. The method according to claim 17, wherein the administering of the inhibitor leads to a decrease of the concentration ratio of monolysocardiolipin (MLCL) to cardiolipin (CL) in a tissue comprised in the subject.

19. The method according to claim 17, wherein the treatment is for the treatment of a disease or condition associated with a mitochondrial disorder.

20. The method according to claim 19, wherein the mitochondrial disorder is a mitochondrial respiratory disorder.

21. The method according to claim 20, wherein the mitochondrial respiratory disorder is Barth syndrome.

22. The method according to claim 17, wherein the treatment is for antiphospholipid syndrome (APS), diabetic cardiomyopathy (DCM), a kidney disorder, an immunological disorder, cancer, Parkinson's disease, ischemia reperfusion injury, heart failure, traumatic brain injury or ageing.


SEQUENCE LISTING (38 sequences)

SEQ ID NO: 1 - protein, 414 aa, Homo sapiens
  REGION 1..414: note = ABHD18 isoform 1
MGVSKLDILYRRLLLTKLFIRGWGRPEDLKRLFEFRKMIGNRERCQNLVSSDYPVHIDKIEEQSDCKILDGHFVSPMAHYVPDIMPIESVIARFQFIVPKEWNSKYRPVCIHLAGTGDHHYWRRRTLMARPMIKEARMASLLLENPYYGCRKPKDQVRSSLKNVSDLFVMGGALVLESAALLHWLEREGYGPLGMTGISMGGHMASLAVSNWPKPMPLIPCLSWSTASGVFTTTDSFKMGQEFVKHFTSSADKLTNLNLVSRTLNLDISNQVVSQKPADCHNSSKTSVSATSEGLLLQDTSKMKRFNQTLSTNKSGYTSRNPQSYHLLSKEQSRNSLRKESLIFMKGVMDECTHVANFSVPVDPSLIIVVQAKEDAYIPRTGVRSLQEIWPGCEIRYLEGGHISAYLFKQGLFR

SEQ ID NO: 2 - protein, 332 aa, Homo sapiens
  REGION 1..332: note = ABHD18 isoform 2
MLDTNLILLPLFQFIVPKEWNSKYRPVCIHLAGTGDHHYWRRRTLMARPMIKEARMASLLLENPYYGCRKPKDQVRSSLKNVSDLFVMGGALVLESAALLHWLEREGYGPLGMTGISMGGHMASLAVSNWPKPMPLIPCLSWSTASGVFTTTDSFKMGQEFVKHFTSSADKLTNLNLVSRTLNLDISNQVVSQKPADCHNSSKTSVSATSEGLLLQDTSKMKRFNQTLSTNKSGYTSRNPQSYHLLSKEQSRNSLRKESLIFMKGVMDECTHVANFSVPVDPSLIIVVQAKEDAYIPRTGVRSLQEIWPGCEIRYLEGGHISAYLFKQGLFR

SEQ ID NO: 3 - protein, 321 aa, Homo sapiens
  REGION 1..321: note = ABHD18 isoform 3
MARPMIKEARMASLLLENPYYGCRKPKDQVRSSLKNVSDLFVMGGALVLESAALLHWLEREGYGPLGMTGISMGGHMASLAVSNWPKPMPLIPCLSWSTASGVFTTGVLSKSINWRELEKQYYTQTVYEEEIIHMLEYCGTDSFKMGQEFVKHFTSSADKLTNLNLVSRTLNLDISNQVVSQKPADCHNSSKTSVSATSEGLLLQDTSKMKRFNQTLSTNKSGYTSRNPQSYHLLSKEQSRNSLRKESLIFMKGVMDECTHVANFSVPVDPSLIIVVQAKEDAYIPRTGVRSLQEIWPGCEIRYLEGGHISAYLFKQGLFR

SEQ ID NO: 4 - protein, 464 aa, Homo sapiens
  REGION 1..464: note = ABHD18 isoform 4
MGVSKLDILYRRLLLTKLFIRGWGRPEDLKRLFEFRKMIGNRERCQNLVSSDYPVHIDKIEEQSDCKILDGHFVSPMAHYVPDIMPIESVIARFQFIVPKEWNSKYRPVCIHLAGTGDHHYWRRRTLMARPMIKEARMASLLLENPYYGCRKPKDQVRSSLKNVSDLFVMGGALVLESAALLHWLEREGYGPLGMTGISMGGHMASLAVSNWPKPMPLIPCLSWSTASGVFTTGVLSKSINWRELEKQYYTQTVYEEEIIHMLEYCGTDSFKMGQEFVKHFTSSADKLTNLNLVSRTLNLDISNQVVSQKPADCHNSSKTSVSATSEGLLLQDTSKMKRFNQTLSTNKSGYTSRNPQSYHLLSKEQSRNSLRKESLIFMKGVMDECTHVANFSVPVDPSLIIVVQAKEDAYIPRTGVRSLQEIWPGCEIRYLEGGHISAYLFKQGLFRQAIYDAFDRFLHKYAN

SEQ ID NO: 5 - protein, 382 aa, Homo sapiens
  REGION 1..382: note = ABHD18 isoform 5
MLDTNLILLPLFQFIVPKEWNSKYRPVCIHLAGTGDHHYWRRRTLMARPMIKEARMASLLLENPYYGCRKPKDQVRSSLKNVSDLFVMGGALVLESAALLHWLEREGYGPLGMTGISMGGHMASLAVSNWPKPMPLIPCLSWSTASGVFTTGVLSKSINWRELEKQYYTQTVYEEEIIHMLEYCGTDSFKMGQEFVKHFTSSADKLTNLNLVSRTLNLDISNQVVSQKPADCHNSSKTSVSATSEGLLLQDTSKMKRFNQTLSTNKSGYTSRNPQSYHLLSKEQSRNSLRKESLIFMKGVMDECTHVANFSVPVDPSLIIVVQAKEDAYIPRTGVRSLQEIWPGCEIRYLEGGHISAYLFKQGLFRQAIYDAFDRFLHKYAN

SEQ ID NO: 6 - protein, 479 aa, Homo sapiens
  REGION 1..479: note = ABHD18 isoform 6
MGVSKLDILYRRLLLTKLFIRGWGRPEDLKRLFEFRKMIGNRERCQNLVSSDYPVHIDKIEEQSDCKILDGHFVSPMAHYVPDIMPIESVIARIIDEMLDTNLILLPLFQFIVPKEWNSKYRPVCIHLAGTGDHHYWRRRTLMARPMIKEARMASLLLENPYYGCRKPKDQVRSSLKNVSDLFVMGGALVLESAALLHWLEREGYGPLGMTGISMGGHMASLAVSNWPKPMPLIPCLSWSTASGVFTTGVLSKSINWRELEKQYYTQTVYEEEIIHMLEYCGTDSFKMGQEFVKHFTSSADKLTNLNLVSRTLNLDISNQVVSQKPADCHNSSKTSVSATSEGLLLQDTSKMKRFNQTLSTNKSGYTSRNPQSYHLLSKEQSRNSLRKESLIFMKGVMDECTHVANFSVPVDPSLIIVVQAKEDAYIPRTGVRSLQEIWPGCEIRYLEGGHISAYLFKQGLFRQAIYDAFDRFLHKYAN

SEQ ID NO: 7 - DNA, 427 bp, synthetic construct
  misc_feature 1..427: note = synthetic DNA fragment
  source 1..427: mol_type = other DNA
cagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacggattaattaaaccgtctcagtttaagagctagaaatagcaagtttaaataaggctagtccgttatcaacttgaaaaagtggcaccgagtcggtgcttttttgaattcgctagctaggtcttgaaaggagtgg

SEQ ID NO: 8 - DNA, 20 bp, Homo sapiens
  misc_feature 1..20: note = sgTAZ#1_exon 2
  source 1..20: mol_type = other DNA
cctgaccgtgcacaacaggg

SEQ ID NO: 9 - DNA, 20 bp, Homo sapiens
  misc_feature 1..20: note = sgTAZ#2_exon 2
  source 1..20: mol_type = other DNA
cctgaccgtgcacaacaggg

SEQ ID NO: 10 - DNA, 20 bp, Homo sapiens
  misc_feature 1..20: note = sgABHD18#1_exon 4
  source 1..20: mol_type = other DNA
caattgaatctgttattgca

SEQ ID NO: 11 - DNA, 20 bp, Homo sapiens
  misc_feature 1..20: note = sgABHD18#2_exon 8
  source 1..20: mol_type = other DNA
atgactggaatatccatggg

SEQ ID NO: 12 - DNA, 75 bp, synthetic construct
  misc_feature 1..75: note = oligonucleotides encoding sgRNAs targeting TAZ or ABHD18
  misc_feature 27..46: note = sgDNA
  source 1..75: mol_type = other DNA
tatcttgtggaaaggacgaaacaccgnnnnnnnnnnnnnnnnnnnngtttaagagctagaaatagcaagtttaaa

SEQ ID NO: 13 - DNA, 21 bp, synthetic construct
  misc_feature 1..21: note = Fwd_sgTAZ#1
  source 1..21: mol_type = other DNA
cacctacagctgcttctggac

SEQ ID NO: 14 - DNA, 21 bp, synthetic construct
  misc_feature 1..21: note = Rev_sgTAZ#1
  source 1..21: mol_type = other DNA
gctgtaagatatgccctgtgg

SEQ ID NO: 15 - DNA, 21 bp, synthetic construct
  misc_feature 1..21: note = Fwd_sgTAZ#2
  source 1..21: mol_type = other DNA
cacctacagctgcttctggac

SEQ ID NO: 16 - DNA, 21 bp, synthetic construct
  misc_feature 1..21: note = Rev_sgTAZ#2
  source 1..21: mol_type = other DNA
gctgtaagatatgccctgtgg

SEQ ID NO: 17 - DNA, 21 bp, synthetic construct
  misc_feature 1..21: note = Fwd_sgABHD18#1
  source 1..21: mol_type = other DNA
aacatccatgacagaccaaga

SEQ ID NO: 18 - DNA, 23 bp, synthetic construct
  misc_feature 1..23: note = Rev_sgABHD18#1
  source 1..23: mol_type = other DNA
tgcaactataaacaatgcttcaa

SEQ ID NO: 19 - DNA, 21 bp, synthetic construct
  misc_feature 1..21: note = Fwd_sgABHD18#2
  source 1..21: mol_type = other DNA
gaggctagaaggtccagctta

SEQ ID NO: 20 - DNA, 21 bp, synthetic construct
  misc_feature 1..21: note = Rev_sgABHD18#2
  source 1..21: mol_type = other DNA
gtaacagcaaaattgggcatt

SEQ ID NO: 21 - DNA, 22 bp, synthetic construct
  misc_feature 1..22: note = 5'Dual-biotin-LAM-PCR
  misc_feature 1: note = 52-Bio
  source 1..22: mol_type = other DNA
ggtctccaaatctcggtggaac

SEQ ID NO: 22 - DNA, 21 bp, synthetic construct
  misc_feature 1..21: note = P7_TG
  source 1..21: mol_type = other DNA
caagcagaagacggcatacga

SEQ ID NO: 23 - DNA, 66 bp, synthetic construct
  misc_feature 1..66: note = MultiplexLinkerBC01_TG
  misc_feature 1: note = 5Phos
  misc_feature 66: note = 3ddC
  source 1..66: mol_type = other DNA
agatcggaagagcacacgtctgaactccagtcacattactcgatctcgtatgccgtcttctgcttg

SEQ ID NO: 24 - DNA, 66 bp, synthetic construct
  misc_feature 1..66: note = MultiplexLinkerBC02_TG
  misc_feature 1: note = 5Phos
  misc_feature 66: note = 3ddC
  source 1..66: mol_type = other DNA
agatcggaagagcacacgtctgaactccagtcactccggagaatctcgtatgccgtcttctgcttg

SEQ ID NO: 25 - DNA, 66 bp, synthetic construct
  misc_feature 1..66: note = MultiplexLinkerBC03_TG
  misc_feature 1: note = 5Phos
  misc_feature 66: note = 3ddC
  source 1..66: mol_type = other DNA
agatcggaagagcacacgtctgaactccagtcaccgctcattatctcgtatgccgtcttctgcttg

SEQ ID NO: 26 - DNA, 66 bp, synthetic construct
  misc_feature 1..66: note = MultiplexLinkerBC04_TG
  misc_feature 1: note = 5Phos
  misc_feature 66: note = 3ddC
  source 1..66: mol_type = other DNA
agatcggaagagcacacgtctgaactccagtcacgagattccatctcgtatgccgtcttctgcttg

SEQ ID NO: 27 - DNA, 56 bp, synthetic construct
  misc_feature 1..56: note = Illumina MultiplexPCRBC01_TG
  source 1..56: mol_type = other DNA
aatgatacggcgaccaccgagatctacacaggctatagatggttctctagcttgcc

SEQ ID NO: 28 - DNA, 56 bp, synthetic construct
  misc_feature 1..56: note = Illumina MultiplexPCRBC02_TG
  source 1..56: mol_type = other DNA
aatgatacggcgaccaccgagatctacacgcctctatgatggttctctagcttgcc

SEQ ID NO: 29 - DNA, 56 bp, synthetic construct
  misc_feature 1..56: note = Illumina MultiplexPCRBC03_TG
  source 1..56: mol_type = other DNA
aatgatacggcgaccaccgagatctacacaggatagggatggttctctagcttgcc

SEQ ID NO: 30 - DNA, 56 bp, synthetic construct
  misc_feature 1..56: note = Illumina MultiplexPCRBC04_TG
  source 1..56: mol_type = other DNA
aatgatacggcgaccaccgagatctacactcagagccgatggttctctagcttgcc

SEQ ID NO: 31 - DNA, 33 bp, synthetic construct
  misc_feature 1..33: note = SolexaSeqFlank_TG (Sequencing primer)
  source 1..33: mol_type = other DNA
ctagcttgccaaacctacaggtggggtctttca

SEQ ID NO: 32 - DNA, 32 bp, synthetic construct
  misc_feature 1..32: note = MultiplexIndex2Read_TG (Sequencing primer)
  source 1..32: mol_type = other DNA
cacctgtaggtttggcaagctagagaaccatc

SEQ ID NO: 33 - DNA, 20 bp, synthetic construct
  misc_feature 1..20: note = forward primer
  source 1..20: mol_type = other DNA
tcctctctggaataaaaaga

SEQ ID NO: 34 - DNA, 20 bp, synthetic construct
  misc_feature 1..20: note = reverse primer
  source 1..20: mol_type = other DNA
catctatagcaaacttcaca

SEQ ID NO: 35 - DNA, 43 bp, synthetic construct
  source 1..43: mol_type = other DNA
  misc_feature 1..43: note = Taz.1 fw FAM primer
agctgaccggcagcaaaattgttttgcttgcgcttttatccca

SEQ ID NO: 36 - DNA, 22 bp, synthetic construct
  source 1..22: mol_type = other DNA
  misc_RNA 1..22: note = Taz.2 rev primer
gcaagaaccgcatgaacagtaa

SEQ ID NO: 37 - RNA, 20 nt, synthetic construct
  source 1..20: mol_type = other RNA
  misc_RNA 1..20: note = taz.target1 sgRNA
cgatgcccttggaggtgaca

SEQ ID NO: 38 - RNA, 20 nt, synthetic construct
  source 1..20: mol_type = other RNA
  misc_feature 1..20: note = taz.target4 sgRNA
gatttcacttactggtccaa